SEQUENCE LISTING (48 sequences)

SEQ ID NO: 1 - DNA, 2297 bp, Homo sapiens
gagccgggctactctgagaagaagacaccaagtggattctgcttcccctgggacagcactgagcgagtgtggagagaggtacagccctcggcctacaagctctttagtcttgaaagcgccacaagcagcagctgctgagccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaaagagatctgttctgggtgttgaccactccagagaagtttcgaggggtcctcacctggttgacccaaaaatgttcccttgaccattggctgcgctaacccccagcccacagagcctgaatttgtaagcaacttgcttctaaatgcccagttcacttctttgcagagccttttacccctgcacagtttagaacagagggaccaaattgcttctaggagtcaactggctggccagtctgggtctgggtttggatctccaattgcctcttgcaggctgagtccctccatgcaaaagtggggctaaatgaagtgtgttaaggggtcggctaagtgggacattagtaactgcacactatttccctctactgagtaaaccctatctgtgattcccccaaacatctggcatggctccctagcattccatgaccagaaacagggacaaagaaatccccccttcagaacagaggcatttaaaatggaaaagagagattggattttggtgggtaacttagaaggatggcatctccatgtagaataaatgaagaaagggaggcccagccgcaggaaggcagaataaatccttgggagtcattaccacgccttgaccttcccaaggttactcagcagcagagagccctgggtgacttcaggtggagagcactagaagtggtttcctgataacaagcaaggatatcagagctgggaaattcatgtggatctggggactgagtgtgggagtgcagagaaagaaagggaaactggctgaggggataccataaaaagaggatgatttcagaaggagaaggaaaaagaaagtaatgccacacattgtgcttggcccctggtaagcagaggctttggggtcctagcccagtgcttctccaacactgaagtgcttgcagatcatctggggacctggtttgaatggagattctgattcagtgggttgggggcagagtttctgcagttccatcaggtcccccccaggtgcaggtgctgacaatactgctgccttacccgccatacattaaggagcagggtcctggtcctaaagagttattcaaatgaaggtggttcgacgccccgaacctcacctgacctcaactaacccttaaaaatgcacacctcatgagtctacctgagcattcaggcagcactgacaatagttatgcctgtactaaggagcatgattttaagaggctttggccaatgcctataaaatgcccatttcgaagatatacaaaaacatacttcaaaaatgttaaacccttaccaacagcttttcccaggagaccatttgtattaccattacttgtataaatacacttcctgcttaaacttgacccaggtggctagcaaattagaaacaccattcatctctaacatatgatactgatgccatgtaaaggcctttaataagtcattgaaatttactgtgagactgtatgttttaattgcatttaaaaatatatagcttgaaagcagttaaactgattagtattcaggcactgagaatgatagtaataggatacaatgtataagctactcacttatctgatacttatttacctataaaatgagatttttgttttccactgtgctattacaaattttcttttgaaagtaggaactcttaagcaatggtaattgtgaataaaaattgatgagagtgttaaaaaaaaaaaaaaaaa

SEQ ID NO: 2 - protein, 155 aa, Homo sapiens
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 3 - DNA, 468 bp, Mus musculus
atggctgaaggggagatcacaaccttcgcagccctgaccgagaggttcaacctgcctctaggaaactacaaaaagcccaaactgctctactgcagcaacgggggccacttcttgaggatccttcctgatggcaccgtggatgggacaagggacaggagcgaccagcacattcagctgcagctcagtgcggaaagtgcgggcgaagtgtatataaagggtacggagaccggccagtacttggccatggacaccgaagggcttttatacggctcgcagacaccaaatgaggaatgtctgttcctggaaaggctggaagaaaaccattataacacttacacctccaagaagcatgcggagaagaactggtttgtgggcctcaagaagaacgggagctgtaagcgcggtcctcggactcactatggccagaaagccatcttgtttctgcccctcccggtgtcttctgactag

SEQ ID NO: 4 - protein, 155 aa, Mus musculus
MAEGEITTFAALTERFNLPLGNYKKPKLLYCSNGGHFLRILPDGTVDGTRDRSDQHIQLQLSAESAGEVYIKGTETGQYLAMDTEGLLYGSQTPNEECLFLERLEENHYNTYTSKKHAEKNWFVGLKKNGSCKRGPRTHYGQKAILFLPLPVSSD

SEQ ID NO: 5 - DNA, 4877 bp, Homo sapiens
actgttgggagaggaatcgtatctccatatttcttctttcagccccaatccaagggttgtagctggaactttccatcagttcttcctttctttttcctctctaagcctttgccttgctctgtcacagtgaagtcagccagagcagggctgttaaactctgtgaaatttgtcataagggtgtcaggtatttcttactggcttccaaagaaacatagataaagaaatctttcctgtggcttcccttggcaggctgcattcagaaggtctctcagttgaagaaagagcttggaggacaacagcacaacaggagagtaaaagatgccccagggctgaggcctccgctcaggcagccgcatctggggtcaatcatactcaccttgcccgggccatgctccagcaaaatcaagctgttttcttttgaaagttcaaactcatcaagattatgctgctcactcttatcattctgttgccagtagtttcaaaatttagttttgttagtctctcagcaccgcagcactggagctgtcctgaaggtactctcgcaggaaatgggaattctacttgtgtgggtcctgcacccttcttaattttctcccatggaaatagtatctttaggattgacacagaaggaaccaattatgagcaattggtggtggatgctggtgtctcagtgatcatggattttcattataatgagaaaagaatctattgggtggatttagaaagacaacttttgcaaagagtttttctgaatgggtcaaggcaagagagagtatgtaatatagagaaaaatgtttctggaatggcaataaattggataaatgaagaagttatttggtcaaatcaacaggaaggaatcattacagtaacagatatgaaaggaaataattcccacattcttttaagtgctttaaaatatcctgcaaatgtagcagttgatccagtagaaaggtttatattttggtcttcagaggtggctggaagcctttatagagcagatctcgatggtgtgggagtgaaggctctgttggagacatcagagaaaataacagctgtgtcattggatgtgcttgataagcggctgttttggattcagtacaacagagaaggaagcaattctcttatttgctcctgtgattatgatggaggttctgtccacattagtaaacatccaacacagcataatttgtttgcaatgtccctttttggtgaccgtatcttctattcaacatggaaaatgaagacaatttggatagccaacaaacacactggaaaggacatggttagaattaacctccattcatcatttgtaccacttggtgaactgaaagtagtgcatccacttgcacaacccaaggcagaagatgacacttgggagcctgagcagaaactttgcaaattgaggaaaggaaactgcagcagcactgtgtgtgggcaagacctccagtcacacttgtgcatgtgtgcagagggatacgccctaagtcgagaccggaagtactgtgaagatgttaatgaatgtgctttttggaatcatggctgtactcttgggtgtaaaaacacccctggatcctattactgcacgtgccctgtaggatttgttctgcttcctgatgggaaacgatgtcatcaacttgtttcctgtccacgcaatgtgtctgaatgcagccatgactgtgttctgacatcagaaggtcccttatgtttctgtcctgaaggctcagtgcttgagagagatgggaaaacatgtagcggttgttcctcacccgataatggtggatgtagccagctctgcgttcctcttagcccagtatcctgggaatgtgattgctttcctgggtatgacctacaactggatgaaaaaagctgtgcagcttcaggaccacaaccatttttgctgtttgccaattctcaagatattcgacacatgcattttgatggaacagactatggaactctgctcagccagcagatgggaatggtttatgccctagatcatgaccctgtggaaaataagatatactttgcccatacagccctgaagtggatagagagagctaatatggatggttcccagcgagaaaggcttattgaggaaggagtagatgtgccagaaggtcttgctgtggactggattggccgtagattctattggacagacagagggaaatctctgattggaaggagtgatttaaatgggaaacgttccaaaataatcactaaggagaacatctctcaaccacgaggaattgctgttcatccaatggccaagagattattctggactgatacagggattaatccacgaattgaaagttcttccctccaaggccttggccgtctggttatagccagctctgatctaatctggcccagtggaataacgattgacttcttaactgacaagttgtactggtgcgatgccaagcagtctgtgattgaaatggccaatctggatggttcaaaacgccgaagacttacccagaatgatgtaggtcacccatttgctgtagcagtgtttgaggattatgtgtggttctcagattgggctatgccatcagtaataagagtaaacaagaggactggcaaagatagagtacgtctccaaggcagcatgctgaagccctcatcactggttgtggttcatccattggcaaaaccaggagcagatccctgcttatatcaaaacggaggctgtgaacatatttgcaaaaagaggcttggaactgcttggtgttcgtgtcgtgaaggttttatgaaagcctcagatgggaaaacgtgtctggctctggatggtcatcagctgttggcaggtggtgaagttgatctaaagaaccaagtaacaccattggacatcttgtccaagactagagtgtcagaagataacattacagaatctcaacacatgctagtggctgaaatcatggtgtcagatcaagatgactgtgctcctgtgggatgcagcatgtatgctcggtgtatttcagagggagaggatgccacatgtcagtgtttgaaaggatttgctggggatggaaaactatgttctgatatagatgaatgtgagatgggtgtcccagtgtgcccccctgcctcctccaagtgcatcaacaccgaaggtggttatgtctgccggtgctcagaaggctaccaaggagatgggattcactgtcttgatattgatgagtgccaactgggggtgcacagctgtggagagaatgccagctgcacaaatacagagggaggctatacctgcatgtgtgctggacgcctgtctgaaccaggactgatttgccctgactctactccaccccctcacctcagggaagatgaccaccactattccgtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgccacgctggccacgggcagcagcagaaggtcatcgtggtggctgtctgcgtggtggtgcttgtcatgctgctcctcctgagcctgtggggggcccactactacaggactcagaagctgctatcgaaaaacccaaagaatccttatgaggagtcgagcagagatgtgaggagtcgcaggcctgctgacactgaggatgggatgtcctcttgccctcaaccttggtttgtggttataaaagaacaccaagacctcaagaatgggggtcaaccagtggctggtgaggatggccaggcagcagatgggtcaatgcaaccaacttcatggaggcaggagccccagttatgtggaatgggcacagagcaaggctgctggattccagtatccagtgataagggctcctgtccccaggtaatggagcgaagctttcatatgccctcctatgggacacagacccttgaagggggtgtcgagaagccccattctctcctatcagctaacccattatggcaacaaagggccctggacccaccacaccaaatggagctgactcagtgaaaactggaattaaaaggaaagtcaagaagaatgaactatgtcgatgcacagtatcttttctttcaaaagtagagcaaaactataggttttggttccacaatctctacgactaatcacctactcaatgcctggagacagatacgtagttgtgcttttgtttgctcttttaagcagtctcactgcagtcttatttccaagtaagagtactgggagaatcactaggtaacttattagaaacccaaattgggacaacagtgctttgtaaattgtgttgtcttcagcagtcaatacaaatagatttttgtttttgttgttcctgcagccccagaagaaattaggggttaaagcagacagtcacactggtttggtcagttacaaagtaatttctttgatctggacagaacatttatatcagtttcatgaaatgattggaatattacaataccgttaagatacagtgtaggcatttaactcctcattggcgtggtccatgctgatgattttgccaaaatgagttgtgatgaatcaatgaaaaatgtaatttagaaactgatttcttcagaattagatggccttattttttaaaatatttgaatgaaaacattttatttttaaaatattacacaggaggccttcggagtttcttagtcattactgtccttttcccctacagaattttccctcttggtgtgattgcacagaatttgtatgtattttcagttacaagattgtaagtaaattgcctgatttgttttcattatagacaacgatgaatttcttctaattatttaaataaaatcaccaaaaacat

SEQ ID NO: 6 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVIRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGVHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 7 - DNA, 4749 bp, Mus musculus
aaaaaaggagaagggattcctatctgtatatagggaaggaatcctatctgcatatttcgttgttagcaccatccctcatcccggtgggcttggaactttccatcaattctttcctgtctcgtttctctttcatcctttgcctggttgtgcctgtctcagggagaaatcagtcacctgcaggccttgcagggctcttaggctctgggaaatttgtcatacgggtgtcaggtacttcttattgctgtccaaagggaaaaaaaaagtgagacaaagaactctcccggagcctttccggctgcactcagaggctctcgagaggtgcagaaggacctggaaaggcagctaaataaaagatgccctggggccgaaggccaacctggctgttgctcgccttcctgctggtgtttttaaagattagcatactcagcgtcacagcatggcagaccgggaactgtcagccaggtcctctcgagagaagcgagagaagcgggacttgtgccggtcctgcccccttcctagttttctcacaaggaaagagcatctctcggattgacccagatggaacaaatcaccagcaattggtggtggatgctggcatctcagcagacatggatattcattataaaaaagagagactctattgggtggatgtagaaagacaagttttgctaagagttttccttaacgggacaggactagagaaagtgtgcaatgtagagaggaaggtgtctgggctggccatagactggatagatgatgaagttctctgggtagaccaacagaacggagtcatcaccgtaacagatatgacagggaaaaattcccgagttcttctaagttccttaaaacatccgtcaaatatagcagtggatccaatagagaggttgatgttttggtcttcagaggtgaccggcagccttcacagagcacacctcaaaggtgttgatgtaaaaacactgctggagacagggggaatatcggtgctgactctggatgtcctggacaaacggctcttctgggttcaggacagtggcgaaggaagccacgcttacattcattcctgtgattatgagggtggctccgtccgtcttatcaggcatcaagcacggcacagtttgtcttcaatggccttttttggtgatcggatcttctactcagtgttgaaaagcaaggcgatttggatagccaacaaacacacggggaaggacacggtcaggattaacctccatccatcctttgtgacacctggaaaactgatggtagtacaccctcgtgcacagcccaggacagaggacgctgctaaggatcctgaccccgaacttctcaaacagaggggaagaccatgccgcttcggtctctgtgagcgagaccccaagtcccactcgagcgcatgcgctgagggctacacgttaagccgagaccggaagtactgcgaagatgtcaatgaatgtgccactcagaatcacggctgtactcttgggtgtgaaaacacccctggatcctatcactgcacatgccccacaggatttgttctgcttcctgatgggaaacaatgtcacgaacttgtttcctgcccaggcaacgtatcaaagtgcagtcatggctgtgtcctgacatcagatggtccccggtgcatctgtcctgcaggttcagtgcttgggagagatgggaagacttgcactggttgttcatcgcctgacaatggtggatgcagccagatctgtcttcctctcaggccaggatcctgggaatgtgattgctttcctgggtatgacctacagtcagaccgaaagagctgtgcagcttcaggaccacagccacttttactgtttgcaaattcccaggacatccgacacatgcattttgatggaacagactacaaagttctgctcagccggcagatgggaatggtttttgccttggattatgaccctgtggaaagcaagatatattttgcacagacagccctgaagtggatagagagggctaatatggatgggtcccagcgagaaagactgatcacagaaggagtagatacgcttgaaggtcttgccctggactggattggccggagaatctactggacagacagtgggaagtctgttgttggagggagcgatctgagcgggaagcatcatcgaataatcatccaggagagaatctcgaggccgcgaggaatagctgtgcatccaagggccaggagactgttctggacggacgtagggatgtctccacggattgaaagcgcttcccttcaaggttccgaccgggtgctgatagccagctccaatctactggaacccagtggaatcacgattgactacttaacagacactttgtactggtgtgacaccaagaggtctgtgattgaaatggccaatctggatggctccaaacgccgaagacttatccagaacgacgtaggtcaccccttctctctagccgtgtttgaggatcacctgtgggtctcggattgggctatcccatcggtaataagggtgaacaagaggactggccaaaacagggtacgtcttcaaggcagcatgctgaagccctcgtcactggttgtggtccatccattggcaaaaccaggtgcagatccctgcttatacaggaatggaggctgtgaacacatctgccaagagagcctgggcacagctcggtgtttgtgtcgtgaaggttttgtgaaggcctgggatgggaaaatgtgtctccctcaggattatccaatcctgtcaggtgaaaatgctgatcttagtaaagaggtgacatcactgagcaactccactcaggctgaagtaccagacgatgatgggacagaatcttccacactagtggctgaaatcatggtgtcaggcatgaactatgaagatgactgtggtcccggggggtgtggaagccatgctcgatgcgtttcagacggagagactgctgagtgtcagtgtctgaaagggtttgccagggatggaaacctgtgttctgatatagatgagtgtgtgctggctagatcggactgccccagcacctcgtccaggtgcatcaacactgaaggtggctacgtctgcagatgctcagaaggctacgaaggagacgggatctcctgtttcgatattgacgagtgccagcggggggcgcacaactgcgctgagaatgccgcctgcaccaacacggagggaggctacaactgcacctgcgcaggccgcccatcctcgcccggacggagttgccctgactctaccgcaccctctctccttggggaagatggccaccatttggaccgaaatagttatccaggatgcccatcctcatatgatggatactgcctcaatggtggcgtgtgcatgcatattgaatcactggacagctacacatgcaactgtgttattggctattctggggatcgatgtcagactcgagacctacgatggtgggagctgcgtcatgctggctacgggcagaagcatgacatcatggtggtggctgtctgcatggtggcactggtcctgctgctcctcttggggatgtgggggacttactactacaggactcggaagcagctatcaaaccccccaaagaacccttgtgatgagccaagcggaagtgtgagcagcagcgggcccgacagcagcagcggggcagctgtggcttcttgtccccaaccttggtttgtggtcctagagaaacaccaagaccccaagaatgggagtctgcctgcggatggtacgaatggtgcagtagtagatgctggcctgtctccctccctgcagctcgggtcagtgcatctgacttcatggagacagaagccccacatagatggaatgggcacagggcaaagctgctggattccaccatcaagtgacagaggaccccaggaaatagagggaaactcccacctaccctcctacagacctgtggggccggagaagctgcattctctccagtcagctaatggatcgtgtcacgaaagggctccagacctgccacggcagacagagccagttaagtagaaactgggagtagacagaaggtacagaagggaaaataacaaaccaggctgatgatggtagagtgctacagacttggtactccagtttccacggctaatcactgctcgctcagggtcctgaagatagctgcacagctgcagagctgcacagcgggatagctgcgacttttgcttcttgctttaagcagttccactgaagatactcaaaagagaagtggagaaaatcattagaaaccaaagtcaagacattcatatataagctgtgtcttcttcactggacggtttgcctcttttccttttgcctcagaaggagtgggttaaagcaggtgaccccatgctctgtcaacccctgaataaatgatgtgatctacatagaagtcttagctcactctcaggaacgcttggaacactataacttttgctatgatatactgccaagtgtggcccatgctcataattgtgccttctgaattgtgataaattagtgaaaaaactgtaacttagaatctgatttattcaggattagatcatctttttatactataaaaatcttcgaatgaaaatatttaactttaaaaacattaccttaatcattgtcttttcttcttgaagtctttcccagtgaaaacgctcaattctgctgtttccatagaatttttaatttattttaagacatgagattgtaaacaaattgcttgatttattttatcctaattatttaaataaaatcaccctaaagcatc

SEQ ID NO: 8 - protein, 1214 aa, Mus musculus
MPWGRRPTWLLLAFLLVFLKISILSVTAWQTGNCQPGPLERSERSGTCAGPAPFLVFSQGKSISRIDPDGTNHQQLVVDAGISADMDIHYKKERLYWVDVERQVLLRVFLNGTGLEKVCNKVSGLAIDWIDDEVLWVDQQNGVITVTDMTGKNSRVLLSSLKHPSNIAVDPIERLMFWSSEVTGSLHRAHLKGVDVKTLLETGGISVLTLDVLDKRLFWVQDSGEGSHAYIHSCDYEGGSVRLIRHQARHSLSSMAFFGDRIFYSVLKSKAIWIANKHTGKDTVRINLHPSFVTPGKLMVVHPRAQPRTEDAAKDPDPELLKQRGRPCRFGLCERDPKSHSSACAEGYTLSRDRKYCEDVNECATQNHGCTLGCENTPGSYHCTCPTGFVLLPDGKQCHELVSCPGNVSKCSHGCVLTSDGPRCICPAGSVLGRDGKTCTGCSSPDNGGCSQICLPLRPGSWECDCFPGYDLQSDRKSCAASGPQPLLLFANSQDIRHMHFDGTDYKVLLSRQMGMVFALDYDPVESKIYFAQTALKWIERANMDGSQRERLITEGVDTLEGLALDWIGRRIYWTDSGKSVVGGSDLSGKHHRIIIQERISRPRGIAVHPRARRLFWTDVGMSPRIESASLQGSDRVLIASSNLLEPSGITIDYLTDTLYWCDTKRSVIEMANLDGSKRRRLIQNDVGHPFSLAVFEDHLWVSDWAIPSVIRVNKRTGQNRVRLQGSMLKPSSLVVVHPLAKPGADPCLYRNGGCEHICQESLGTARCLCREGFVKAWDGKMCLPQDYPILSGENADLSKEVTSLSNSTQAEVPDDDGTESSTLVAEIMVSGMNYEDDCGPGGCGSHARCVSDGETAECQCLKGFARDGNLCSDIDECVLARSDCPSTSSRCINTEGGYVCRCSEGYEGDGISCFDIDECQRGAHNCAENAACTNTEGGYNCTCAGRPSSPGRSCPDSTAPSLLGEDGHHLDRNSYPGCPSSYDGYCLNGGVCMHIESLDSYTCNCVIGYSGDRCQTRDLRWWELRHAGYGQKHDIMVVAVCMVALVLLLLLGMWGTYYYRTRKQLSNPPKNPCDEPSGSVSSSGPDSSSGAAVASCPQPWFVVLEKHQDPKNGSLPADGTNGAVVDAGLSPSLQLGSVHLTSWRQKPHIDGMGTGQSCWIPPSSDRGPQEIEGNSHLPSYRPVGPEKLHSLQSANGSCHERAPDLPRQTEPVK

SEQ ID NO: 9 - DNA, 1201 bp, Homo sapiens
taggcactgactccgaacaggattctttcacccaggcatctcctccagagggatccgccagcccgtccagcagcaccatgtgggtgaccaaactcctgccagccctgctgctgcagcatgtcctcctgcatctcctcctgctccccatcgccatcccctatgcagagggacaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctttttgccttcgagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgagacataacatgggctctcaactgatggtgaacttcttctggtgagtgacagaggctgcagtgaagaataatgagtctaatagaagtttatcacagatgtctctaatctctatagctgatccctacctctctcgctgtctttgtacccagcctgcattctgtttcgatctgtcttttagcagtccatacaatcatttttctacatgctggcccttacccagcttttctgaatttacaataaaaactattttttaacgtg

SEQ ID NO: 10 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 11 - DNA, 2204 bp, Mus musculus
atgatgtgggggaccaaacttctgccggtcctgttgctgcagcatgtcctcctgcacctcctcctgcttcatgtcgccatcccctatgcagaaggacagaagaaaagaagaaatacacttcatgaatttaaaaagtcagcaaaaactactcttaccaaggaagacccattactgaagattaaaaccaaaaaagtgaactctgcagatgagtgtgccaacaggtgtatcaggaacaggggctttacgttcacttgcaaggccttcgtttttgataagtcaagaaaacgatgctactggtatcctttcaatagtatgtcaagtggagtgaaaaaagggtttggccatgaatttgacctctatgaaaacaaagactatattagaaactgcatcattggtaaaggaggcagctataaagggacggtatccatcactaagagtggcatcaaatgccagccttggaattccatgatcccccatgaacacagctttttgccttcgagctatcgcggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggtgaaagctacagaggtcccatggatcacacagaatcaggcaagacttgtcagcgctgggaccagcagacaccacaccggcacaagttcttgccagaaagatatcccgacaagggctttgatgataattattgccgcaatcctgatggcaagccgaggccatggtgctacactcttgaccctgacaccccttgggagtattgtgcaattaaaacgtgcgctcacagtgctgtgaatgagactgatgtccctatggaaacaactgaatgcattcaaggccaaggagaaggttacaggggaaccagcaataccatttggaatggaattccctgtcagcgttgggattcgcagtaccctcacaagcatgatatcactcccgagaacttcaaatgcaaggaccttagagaaaattattgccgcaatccagatggggctgaatcaccatggtgttttaccactgacccaaacatccgagttggctactgctctcaaattcccaagtgtgacgtgtcaagtggacaagattgttatcgtggcaatgggaaaaattacatgggcaacttatccaaaacaaggtctggacttacatgttccatgtgggacaagaatatggaggatttacaccgtcatatcttctgggagccagatgctagcaaattgaataagaattactgccggaatcctgatgatgatgcccatggaccttggtgctacacggggaatcctcttattccttgggattattgccctatttcccgttgtgaaggagatactacacctacaattgtcaatttggaccatcctgtaatatcctgtgccaaaacaaaacaactgcgggttgtaaatggcattccaacacaaacaacagtagggtggatggttagtttgaaatacagaaataaacatatctgtggaggatcattgataaaggaaagttgggttcttactgcaagacaatgttttccagccagaaacaaagacttgaaagactatgaagcttggcttggcatccacgatgttcatgagagaggcgaggagaagcgcaagcagatcttaaacatttcccagctggtctatggtcctgaaggctcagacttggttttactgaagcttgctcgacctgcaatcctggataactttgtcagtacaattgatttacctagttatggttgtacaatccctgaaaagaccacttgcagtatttacggctggggctacactggattgatcaacgcggatggtttattacgagtagctcatctgtatattatggggaatgagaaatgcagtcagcaccatcaaggcaaggtgactttgaatgagtctgagttatgtgctggggctgaaaagattggatcaggaccatgtgagggagattatggtggcccactcatttgtgaacaacacaaaatgagaatggttcttggtgtcattgttcctggtcgtggatgtgccatcccaaatcgtcctggtatttttgttcgagtagcatattatgcaaaatggatacacaaagtaattttgacatacaagttgtaatagccatagaagaggcc

SEQ ID NO: 12 - protein, 727 aa, Mus musculus
MWGTKLLPVLLLQHVLLHLLLLHVAIPYAEGQKKRRNTLHEFKKSAKTTLTKEDPLLKIKTKKVNSADECANRCIRNRGFTFTCKAFVFDKSRKRCYWYPFNSMSSGVKKGFGHEFDLYENKDYIRNCIIGKGGSYKGTVSITKSGIKCQPWNSMIPHEHSFLPSSYRGKDLQENYCRNPRGEEGGPWCFTSNPEVRYEVCDIPQCSEVECMTCNGESYRGPMDHTESGKTCQRWDQQTPHRHKFLPERYPDKGFDDNYCRNPDGKPRPWCYTLDPDTPWEYCAIKTCAHSAVNETDVPMETTECIQGQGEGYRGTSNTIWNGIPCQRWDSQYPHKHDITPENFKCKDLRENYCRNPDGAESPWCFTTDPNIRVGYCSQIPKCDVSSGQDCYRGNGKNYMGNLSKTRSGLTCSMWDKNMEDLHRHIFWEPDASKLNKNYCRNPDDDAHGPWCYTGNPLIPWDYCPISRCEGDTTPTIVNLDHPVISCAKTKQLRVVNGIPTQTTVGWMVSLKYRNKHICGGSLIKESWVLTARQCFPARNKDLKDYEAWLGIHDVHERGEEKRKQILNISQLVYGPEGSDLVLLKLARPAILDNFVSTIDLPSYGCTIPEKTTCSIYGWGYTGLINADGLLRVAHLYIMGNEKCSQHHQGKVTLNESELCAGAEKIGSGPCEGDYGGPLICEQHKMRMVLGVIVPGRGCAIPNRPGIFVRVAYYAKWIHKVILTYKL

SEQ ID NO: 13 - DNA, 618 bp, Homo sapiens
atggcggcgcccggcgagcggggccgcttccacggcgggaacctcttcttcctgccggggggcgcgcgctccgagatgatggacgacctggcgaccgacgcgcggggccggggcgcggggcggagagacgcggccgcctcggcctcgacgccagcccaggcgccgacctccgattctcctgtcgccgaggacgcctcccggaggcggccgtgccgggcctgcgtcgacttcaagacgtggatgcggacgcagcagaagcgggacaccaagtttagagaggactgcccgccggatcgcgaggaactgggccgccacagctgggctgtcctccacaccctggccgcctactaccccgacctgcccaccccagaacagcagcgagacatggcccagttcatacatttattttctaagttttacccctgtgaggagtgtgctgaagacctaagaaaaaggttgtgcaggaaccacccagacacccgcacccgggcatgcttcacacagtggctgtgccacctgcacaatgaagtgaaccgcgagctgggcaagcctgacttcgactgctcaaaagtggatgagcgctggcgcgacggctggaaggatggctcctgtgactag

SEQ ID NO: 14 - protein, 275 aa, Homo sapiens
MISTSWGAPKAFSKGFNLQHVADGLYGSHLHVYSWPGGEIKQLIDLGPTGLLPLEIRFLHDPSKDTGFVGSALSSNMIRFFKNSDETWSHEVVISVKPLKVENWILPEMPGLITDFLISLDDRFIYFVNWLHGDIRQYNIEDPKNPVLTGQIWVGGLLQKGSPVKAVGEDGNTFQFEVPQIKGKSLRGGPQMIQLSLDGKRLYATNSLFSAWDRQFYPEIMEKGSHIIQIDVDTEKGGLTINPDFFVDFGDEPDGPSLAHEMRYPGGDCTSDIWI

SEQ ID NO: 15 - DNA, 559 bp, Mus musculus
cgacaccaccccttccgcggccccggcgccgcaaggtttggagcacgggaagcgaccgtgccgggcctgcgtggacttcaagtcgtggatgcggacccagcagaagcgggacatcaagtttagggaggactgtccgcaggatcgggaagaattgggtcgccacacctgggctttcctccatacgctggccgcctattacccggacaggcccacgccagaacaacaacaggatatggcccagttcatacatatattttccaagttttacccctgcgaggaatgtgcggaagacataaggaagaggataggcaggaaccagccagacacaagcactcgagtatccttcagccagtggctgtgccgcctgcacaatgaggtgaatcggaagctgggcaagcctgattttgactgctcgagagtagatgagcgttggcgtgacggatggaaggacggctcctgtgactagaagattaccagcagttcgggagggggatctaggctggttctatgggcaacagcctgattgacgattaaagtgcatctgagccaacacttgttt

SEQ ID NO: 16 - protein, 125 aa, Mus musculus
MRTQQKRDIKFREDCPQDREELGRHTWAFLHTLAAYYPDRPTPEQQQDMAQFIHIFSKFYPCEECAEDIRKRIGRNQPDTSTRVSFSQWLCRLHNEVNRKLGKPDFDCSRVDERWRDGWKDGSCD

SEQ ID NO: 17 - DNA, 600 bp, Mus musculus
atggcggcgcccagcgagccggcgggcttccctcgcggcagtcgcttctccttcctgccgggcggcgcgcgctccgagatgaccgacgacctggtgactgacgcgcggggccgcggcgcaaggcatagagacgacaccaccccttccgcggccccggcgccgcaaggtttggagcacgggaagcgaccgtgccgggcctgcgtggacttcaagtcgtggatgcggacccagcagaagcgggacatcaagtttagggaggactgtccgcaggatcgggaagaattgggtcgccacacctgggctttcctccatacgctggccgcctattacccggacaggcccacgccagaacaacaacaggatatggcccagttcatacatatattttccaagttttacccctgcgaggaatgtgcggaagacataaggaagaggataggcaggaaccagccagacacaagcactcgagtatccttcagccagtggctgtgccgcctgcacaatgaggtgaatcggaagctgggcaagcctgattttgactgctcgagagtagatgagcgttggcgtgacggctggaaggacggctcctgtgactagtga

SEQ ID NO: 18 - protein, 198 aa, Mus musculus
MAAPSEPAGFPRGSRFSFLPGGARSEMTDDLVTDARGRGARHRDDTTPSAAPAPQGLEHGKRPCRACVDFKSWMRTQQKRDIKFREDCPQDREELGRHTWAFLHTLAAYYPDRPTPEQQQDMAQFIHIFSKFYPCEECAEDIRKRIGRNQPDTSTRVSFSQWLCRLHNEVNRKLGKPDFDCSRVDERWRDGWKDGSCD

SEQ ID NO: 19 - DNA, 1869 bp, Homo sapiens
gtcacccccagcgggcgcgggccggagcacgggcacccagcatgggggtactgctcacacagaggacgctgctcagtctggtccttgcactcctgtttccaagcatggcgagcatggcggctataggcagctgctcgaaagagtaccgcgtgctccttggccagctccagaagcagacagatctcatgcaggacaccagcagactcctggacccctatatacgtatccaaggcctggatgttcctaaactgagagagcactgcagggagcgccccggggccttccccagtgaggagaccctgagggggctgggcaggcggggcttcctgcagaccctcaatgccacactgggctgcgtcctgcacagactggccgacttagagcagcgcctccccaaggcccaggatttggagaggtctgggctgaacatcgaggacttggagaagctgcagatggcgaggccgaacatcctcgggctcaggaacaacatctactgcatggcccagctgctggacaactcagacacggctgagcccacgaaggctggccggggggcctctcagccgcccacccccacccctgcctcggatgcttttcagcgcaagctggagggctgcaggttcctgcatggctaccatcgcttcatgcactcagtggggcgggtcttcagcaagtggggggagagcccgaaccggagccggagacacagcccccaccaggccctgaggaagggggtgcgcaggaccagaccctccaggaaaggcaagagactcatgaccaggggacagctgccccggtagcctcgagagcaccccttgccggtgaaggatgcggcaggtgctctgtggatgagaggaaccatcgcaggatgacagctcccgggtccccaaacctgttcccctctgctactagccactgagaagtgcactttaagaggtgggagctgggcagacccctctacctcctccaggctgggagacagagtcaggctgttgcgctcccacctcagccccaagttccccaggcccagtggggtggccgggcgggccacgcgggaccgactttccattgattcaggggtctgatgacacaggctgactcatggccgggctgactgcccccctgccttgctccccgaggcctgccggtccttccctctcatgacttgcagggccgttgcccccagacttcctcctttccgtgtttctgaaggggaggtcacagcctgagctggcctcctatgcctcatcatgtcccaaaccagacacctggatgtctgggtgacctcactttaggcagctgtaacagcggcagggtgtcccaggagccctgatccgggggtccagggaatggagctcaggtcccaggccagccccgaagtcgccacgtggcctggggcaggtcactttacctctgtggacctgttttctctttgtgaagctagggagttagaggctgtacaaggcccccactgcctgtcggttgcttggattccctgacgtaaggtggatattaaaaatctgtaaatcaggacaggtggtgcaaatggcgctgggaggtgtacacggaggtctctgtaaaagcagacccacctcccagcgccgggaagcccgtcttgggtcctcgctgctggctgctccccctggtggtggatcctggaattttctcacgcaggagccattgctctcctagagggggtctcagaaactgcgaggccagttccttggagggacatgactaatttatcgatttttatcaatttttatcagttttatatttataagccttatttatgatgtatatttaatgttaatattgtgcaaacttatatttaaaacttgcctggtttctaaaaaaaaaaaaaaaaaa

SEQ ID NO: 20 - protein, 252 aa, Homo sapiens
MGVLLTQRTLLSLVLALLFPSMASMAAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPNRSRRHSPHQALRKGVRRTRPSRKGKRLMTRGQLPR

SEQ ID NO: 21 - DNA, 1848 bp, Mus musculus
gtcacccctgagaggcacgggccagagtaccaggacccagtatgcagacacggcttctaagaacactgctcagtttgaccctcagtctcctcatcctgagcatggcactggccaatcgtggctgctccaactcttcctctcagctcctcagccagctgcagaatcaggcgaacctcacggggaacacagaatcactcttggagccctatatccgcctccaaaacctgaacacacctgacctgagagctgcctgcacccagcactctgtggccttccccagtgaggacacactccggcaactgagcaagcctcacttcctgagcactgtgtacaccacactggacagagtcttgtaccaactggatgctttaagacagaaatttctgaagactccggcttttccaaagctggacagtgcccggcacaatatcctcggcataaggaacaatgttttctgcatggcccggctgctcaaccactccctggagatacctgagcccacacagacagactctggggcctcacggtccactacaacaccagatgtctttaataccaagataggcagctgtggctttctctggggataccatcgcttcatgggctcagtggggagggtcttcagggaatgggacgatggctccacacgcagccggagacagagcccgctccgggcccggcgcaagggaacccgcagaatccgggtccggcacaagggaacccgcagaatccgggtccggcgcaagggaacccgcagaatctgggtccggcgcaagggatcccgcaaaatcagaccttccaggagcacccagagcccgacgaccagggcctaggttccctggtagcctgaggacacactgacagacagcatagtctggtgatacaggatgtcgctctcagaggctttcaaagctgcttctgtcaccaggggtcacacagaagagcactttaaggggtgaagttgagtgtcccctactaccactcaggacttcaaggatagtgaggttattgtgtccccactccaagcctccagtcctagtggggtggctgggtcggaccacgtggggccggaggttttccattgattcaggggtctgatgacacaagctgattcaccacagggctggctgggctgaacccctcgggctgttggtcctttcctctcatgacttgaaactgtttcctccagacttcctcctttccctgtggctgggttccaaagagaggtctgatccggtgctctctctcatgccttatcccactcaggacagatacctggacctctgggtgacctcacacttggcagttgcgacaggggcagggtgtcctaccaaggaacactgatctgggcgttcagggaagagagctcagagcctagcttgttccctaattctctgtgtgactgtgagcaagacactttatttatccgaatgtcagcgttctctgtggaaagctgtgttgtgtgtgtgggtccttaggtaatagccccctctgcctgtcagctgctggactctgacatagggtggacatcaaagtctctgtaaatgggaacctgtggtgcaaacggttgtggggtgtgtttatgggagatctcccagtgcctaaaagccctgttttgggtcctcgctgcatgatgctccctctggtgatgtgttgtgaaatttttcacaggctgaaccagtcctcttgaaaggtctcagaagctggtgagcaattacttggagggacatgactaatttattgttttattttttatcagtttaatccgttttatatttataaggcctatttataatgtatatttaatgttaatattttgctaacatatttaaaacctgtcttgtttct

SEQ ID NO: 22 - protein, 263 aa, Mus musculus
MQTRLLRTLLSLTLSLLILSMALANRGCSNSSSQLLSQLQNQANLTGNTESLLEPYIRLQNLNTPDLRAACTQHSVAFPSEDTLRQLSKPHFLSTVYTTLDRVLYQLDALRQKFLKTPAFPKLDSARHNILGIRNNVFCMARLLNHSLEIPEPTQTDSGASRSTTTPDVFNTKIGSCGFLWGYHRFMGSVGRVFREWDDGSTRSRRQSPLRARRKGTRRIRVRHKGTRRIRVRRKGTRRIWVRRKGSRKIRPSRSTQSPTTRA

SEQ ID NO: 23 - DNA, 33 bp, Mus musculus
tattcatatgcggacccagcagaagcgggacat

SEQ ID NO: 24 - DNA, 31 bp, Mus musculus
ttatcactagtcacaggagccgtccttccat

SEQ ID NO: 25 - DNA, 32 bp, Mus musculus
tcactagtcacaggagccgtccttccatccgt

SEQ ID NO: 26 - DNA, 424 bp, Mus musculus
catatgcggacccagcagaagcgggacatcaagtttagggaggactgtccgcaggatcgggaagaattgggtcgccacacctgggctttcctccatacgctggccgcctattacccggacaggcccacgccagaacaacaacaggatatggcccagttcatacatatattttccaagttttacccctgcgaggaatgtgcggaagacataaggaagaggataggcaggaaccagccagacacaagcactcgagtatccttcagccagtggctgtgccgcctgcacaatgaggtgaatcggaagctgggcaagcctgattttgactgctcgagagtagatgagcgttggcgtgacggatggaaggacggctcctgtgactagtgaaagggcgaattctgcagatatccatcacactggcggccgc

SEQ ID NO: 27 - DNA, 22 bp, Mus musculus
cagccaaagtggagtggaaaga

SEQ ID NO: 28 - DNA, 21 bp, Mus musculus
aactctcggcaggttctggaa

SEQ ID NO: 29 - DNA, 22 bp, Mus musculus
attgagaagacccctgccttgt

SEQ ID NO: 30 - DNA, 21 bp, Mus musculus
atctgcaatgtgtcagccagc

SEQ ID NO: 31 - DNA, 22 bp, Mus musculus
attgagaagacccctgccttgt

SEQ ID NO: 32 - DNA, 21 bp, Mus musculus
atctgcaatgtgtcagccagc

SEQ ID NO: 33 - DNA, 22 bp, Mus musculus
cctgaaccaatcccacctctct

SEQ ID NO: 34 - DNA, 21 bp, Mus musculus
atctcccgttgctttctgacg

SEQ ID NO: 35 - DNA, 22 bp, Mus musculus
attgagaagacccctgccttgt

SEQ ID NO: 36 - DNA, 21 bp, Mus musculus
atctgcaatgtgtcagccagc

SEQ ID NO: 37 - DNA, 21 bp, Mus musculus
aagaagatggctttcaggccc

SEQ ID NO: 38 - DNA, 21 bp, Mus musculus
aaggccattgaagtgtggtgg

SEQ ID NO: 39 - DNA, 22 bp, Mus musculus
gactctctaaaacccttgccgg

SEQ ID NO: 40 - DNA, 21 bp, Mus musculus
ccatggtcaacacctgcacat

SEQ ID NO: 41 - DNA, 22 bp, Mus musculus
cgcccatcggtataatgatttg

SEQ ID NO: 42 - DNA, 22 bp, Mus musculus
ctgcactaatttggcatgctca

SEQ ID NO: 43 - DNA, 20 bp, Mus musculus
tgcttgatgtgcaccattgc

SEQ ID NO: 44 - DNA, 21 bp, Mus musculus
tgctccagatgctgcatcttc

SEQ ID NO: 45 - DNA, 21 bp, Mus musculus
aagcagagtggaccaaccgtt

SEQ ID NO: 46 - DNA, 21 bp, Mus musculus
aagcagagtggaccaaccgtt

SEQ ID NO: 47 - DNA, 20 bp, Mus musculus
ttaatgtgcttggcccgatc

SEQ ID NO: 48 - DNA, 22 bp, Mus musculus
ccagcgaaggcttgttttagaa

(19) United States
(12) Patent Application Publication
(10) Pub. No.: US 2004/0191902 A1
(43) Pub. Date: Sep. 30, 2004

(54) Growth and differentiation of stem cells

(75) Inventors: John E. Hambor, Madison, CT (US); Marsha L. Roach, Waterford, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/813,805

(22) Filed: Mar. 30, 2004

Related U.S. Application Data
(60) Provisional application No. 60/459,449, filed on Mar. 31, 2003.

(51) Int. Cl. C12N 5/08

(52) U.S. Cl. 435/370

(57) ABSTRACT

The present invention relates to methods of culturing stem cells to produce hepatocyte-like cells. Among other advances, the invention also relates to purified preparations of hepatocyte-like cells and methods for using the hepatocyte-like cells.

This application claims priority, under 35 U.S.C. §119(e), from U.S. provisional application 60/459,449 filed Mar. 31, 2003.

### FIELD OF THE INVENTION

The present invention relates to methods of culturing stem cells to produce hepatocyte-like cells. The invention also relates to purified preparations of hepatocyte-like cells and methods for using hepatocyte-like cells.

### BACKGROUND OF THE INVENTION

The liver participates in many important physiological processes, including protein, lipid and carbohydrate metabolism, bile secretion, fibrinogen production and detoxification of a wide variety of foreign compounds and endogenous metabolites, including many therapeutic agents. By virtue of a portal circulatory system, the liver is the initial processing point for most materials absorbed through the gastrointestinal tract, and in this manner the liver protects the body from many harmful compounds. Hepatocytes are the most significant type of parenchymal cell in the liver, and hepatocytes perform most of the liver functions mentioned above.

In drug development, great significance is attached to the nature of the interaction between a candidate therapeutic and the cells of the liver. Many candidate therapeutics are significantly hepatotoxic. In addition, the pharmacokinetics of a candidate therapeutic are heavily influenced by the metabolic activities of hepatocytes. In vitro assays for predicting the in vivo hepatotoxicity and pharmacokinetics of a candidate compound are an important part of the drug development process.

Clinically, a ready source of metabolically active and transplantable liver cells would be invaluable. The liver is vulnerable to many disorders. A variety of compounds can cause temporary or permanent liver failure as well as liver cell death. In addition, a variety of diseases, such as hepatitis, may result in liver damage. While these conditions are usually correctable with a liver transplant, the chronic shortage of donor organs places this method of treatment out of reach for many patients.

Hepatocytes cultured from liver samples are useful for investigating the interaction between candidate therapeutics and the liver. However such cells are difficult to obtain in large numbers and cannot be maintained in culture as a stable, genetically uniform cell line. In addition, primary hepatocytes have not been successfully used to restore liver function in a subject with a damaged liver, partly because they are difficult to obtain in sufficient quantities.

For these and other reasons, it would be beneficial to have an alternate source for cells that have the characteristics of hepatocytes.

### SUMMARY OF THE INVENTION

The present invention relates generally to methods of culturing stem cells to produce hepatocyte-like cells. The invention also relates to purified preparations of hepatocyte-like cells and methods for using hepatocyte-like cells.

In a first embodiment, the invention relates to methods for obtaining a hepatocyte-like cell, comprising providing a stem cell, culturing the stem cell in a first medium comprising effective amounts of an acidic fibroblast growth factor (aFGF) and an epidermal growth factor (EGF) for about 2 to 4 days to obtain a first cell population, culturing a cell of the first cell population in a second medium comprising an effective amount of hepatocyte growth factor (HGF) for about 2 to 4 days to obtain a second cell population, and culturing a cell of the second cell population in a third medium comprising effective amounts of oncostatin-M for about 2 to 4 days to obtain a third cell population, the third cell population comprising a plurality of hepatocyte-like cells.

In a second embodiment, the invention relates to methods for obtaining a hepatocyte-like cell, comprising providing an ES cell, stimulating the differentiation of the ES cell into embryoid bodies for about 5 days, culturing the embryoid bodies in a first medium comprising effective amounts of an aFGF and an EGF for about 1 to 2 days to obtain embryoid bodies, dissociating the embryoid bodies into a single cell suspension and culturing the single cell suspension for about 1 to 2 days in the first medium to obtain a first cell population, culturing a cell of the first cell population in a second medium comprising an effective amount of EGF, HGF and aFGF for about 2 to 4 days to obtain a second cell population, and culturing a cell of the second cell population in a third medium comprising effective amounts of oncostatin-M, EGF, and HGF for about 2 to 4 days to obtain a third cell population, the third cell population comprising a plurality of hepatocyte-like cells.

In a third embodiment, the invention relates to methods for obtaining a cellular composition comprising an enriched population of hepatocyte-like cells, comprising providing a cellular composition comprising a plurality of hepatocyte-like cells, and culturing the cellular composition in a medium suitable for selectively culturing gluconeogenic cells, thereby obtaining a cellular composition comprising an enriched population of hepatocyte-like cells.

In a fourth embodiment, the invention relates to cellular compositions comprising viable cells, wherein at least 90% of the viable cells are hepatocyte-like cells.

In a fifth embodiment, the invention relates to methods for determining whether a test agent is toxic to a hepatic cell, comprising contacting a hepatocyte-like cell according to the invention with the test agent for a time sufficient for any toxic effect on the cell to be detected, and determining the toxic effect on the cell.

In a sixth embodiment, the invention relates to methods for identifying a metabolic product of a test agent, comprising contacting a hepatocyte-like cell according to the invention with the test agent for a time sufficient for the test agent to be metabolized, and detecting the presence of a metabolized product.

In a seventh embodiment, the invention relates to methods for treating a subject in need of liver cells, comprising administering to the subject a therapeutically effective amount of the hepatocyte-like cells according to the invention.

In a eighth embodiment, the invention relates to isolated nucleic acids encoding a polypeptide having SEQ ID NO: 18.

In a ninth embodiment, the invention relates to isolated polypeptides comprising the amino acid sequence SEQ ID NO: 18.

In a tenth embodiment, the invention relates to methods for stimulating the proliferation of a hepatocyte-like cell or precursor thereof, comprising contacting a cellular composition according to the invention with an isolated polypeptide comprising the amino acid sequence set forth in SEQ ID NO: 18.

### DETAILED DESCRIPTION OF THE INVENTION

The invention is based in part on the discovery that embryonic stem cells can be differentiated into a highly purified population of hepatocyte-like cells.

1. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “acidic fibroblast growth factor” or “aFGF” includes the native aFGF from any organism as well as any functional mimic thereof. An exemplary nucleotide sequence of human aFGF is set forth in SEQ ID NO: 1 and the protein encoded thereby is set forth in SEQ ID NO: 2, which sequences correspond to GenBank® Accession Numbers NM_{—}000800 and NP_{—}000791.1, respectively. Other spliceforms of aFGF are set forth in GenBank® Accession Numbers NM_{—}033137 and NM_{—}033136. An exemplary nucleotide sequence of a mouse aFGF is set forth in SEQ ID NO: 3 and the protein encoded thereby is set forth in SEQ ID NO: 4, which sequences correspond to GenBank® Accession Numbers M30641 and AAA37618.1, respectively.

“Agent” refers to a chemical compound, such as small molecules and biological macromolecules (e.g., DNA, RNA, polypeptides or lipids).

“Cells” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The terms “cell culture” or “culture” include any combination of cells and medium. The cells need not be actively growing.

A “cellular composition” is a composition that comprises a plurality of viable cells, wherein the viable cells are not in their natural context. For example, cellular compositions do not include whole organisms. Cellular compositions may comprise materials (e.g. media, support matrix, dead cells, pharmaceutically acceptable carriers etc.) in addition to the plurality of viable cells. Exemplary cellular compositions include liquid or plated cell cultures, cell suspension, cells seeded on a matrix, artificial tissue constructs, frozen cells, cells prepared with a suitable carrier for administration to a subject, etc. A viable cell is a cell that is capable of performing the metabolic functions of a cell when placed in the appropriate conditions. For example, a viable cell may be an actively growing cell, a cell no longer capable of undergoing cell division but nonetheless metabolically active, or a frozen cell that is not metabolically active but may become metabolically active when thawed in the appropriate conditions.

A “cell population” is a plurality of cells.

An “embryonic stem cell” or “ES cell” refers to a totipotent stem cell isolated from the inner cell mass of an early stage blastocyst, as described, e.g., in E. J. Robertson “Embryo-derived stem cell lines, in Teratocarcinomas and embryonic stem cells: a practical approach, E. J. Robertson, editor, IRL Press, Washington D.C., 1987.

“Endogenous”, in reference to a growth factor or other substance, refers to the fact that the substance is in a form substantially similar to a form found in nature or a form predicted to be found in nature (i.e., a polypeptide including predicted glycosylation structures, even if such glycosylation structures have not been characterized in a form of the substance found in nature).

The term “epidermal growth factor” or “EGF” includes the native EGF from any organism, as well as functional mimics. An exemplary nucleotide sequence of human EGF is set forth in SEQ ID NO: 5 and the protein encoded thereby is set forth in SEQ ID NO: 6, which sequences correspond to GenBank® Accession Numbers NM_{—}001963 and NP_{—}001954.1, respectively. An exemplary nucleotide sequence of a mouse EGF is set forth in SEQ ID NO: 7 and the protein encoded thereby is set forth in SEQ ID NO: 8, which sequences correspond to GenBank® Accession Numbers J00380 and AAA37539.1, respectively.

“Fragment” as used in reference to a factor, e.g., a growth factor, includes polypeptides that have an amino-terminal and/or carboxy-terminal deletion relative to a naturally occurring form of the factor. An “active fragment” is a fragment that retains at least enough functional activity of the relevant factor that the active fragment may be used as a replacement for the factor. Optionally, an active fragment retains 25% or more of the activity of the relevant factor.

The term “gluconeogenic” used in reference to a cell refers to any cell that is capable of generating glucose from a simpler molecule than glucose, such as pyruvate, lactate, amino acids, glycerol or propionate. In mammals, gluconeogenic cells generally express some or all of the gluconeogenic enzymes: glucose-6-phosphatase, fructose-1,6-bisphosphatase, pyruvate carboxylase and pyruvate carboxykinase. In humans, only two cell types are known to be gluconeogenic: breast epithelium and hepatocytes.

A “hepatocyte-like cell” is a cell having a plurality, e.g., at least two, of characteristics of a hepatocyte. Exemplary hepatocyte-like cells include cells having two or more of the following properties: the ability to use pyruvate as a sole carbon source; butyrate resistance at concentrations of at least about 1-20 mM and preferably at least about 5 mM sodium butyric acid; the ability to take up vinblastine; cytochrome P450 activity (e.g., dibenzylfluorescein metabolism, dextromethorphan oxidation, coumarin glucaronidation or sulfation); cytochrome P450 expression (detected, for example, by RT-PCR or antibody staining); or expression of other genes and/or proteins that are indicative of hepatocytes, such as α-fetoprotein, γ-glutyryltransferase, hepatocyte nuclear factor (HNF) 1α, HNF 1β, HNF 3α, HNF 3β, HNF 4, anti-trypsin, transthyretin, and CFTR. Hepatocyte-like cells may be immortalized or aneuploid, but need not be.

The term “hepatocyte growth factor” or “HGF” includes the native HGF from any organism, as well as functional mimics. An exemplary nucleotide sequence of human HGF is set forth in SEQ ID NO: 9 and the protein encoded thereby is set forth in SEQ ID NO: 10, which sequences correspond to GenBank® Accession Numbers M29145 and AAA52650.1, respectively. An exemplary nucleotide sequence of a mouse HGF is set forth in SEQ ID NO: 11 and the protein encoded thereby is set forth in SEQ ID NO: 12, which sequences correspond to GenBank® Accession Numbers D10212 and BAA01064.1, respectively.

The term “hepatopoietin” or “HPO” includes the native HPO from any organism, as well as functional mimics. Human HPO is a polypeptide mitogen that is described as consisting of a 15.1 kDa protein of 206 amino acids (see, e.g., Wang et al., J. Biol. Chem., 274:11469 (1999), Yang et al., Sci. China Ser. C Life Sci., 40:642 (1997), and GenBank® Accession No. AF306863). Another human HPO is encoded by the nucleotide sequence set forth in GenBank® Accession number AF183892, encoding a protein of 180 amino acids having the sequence set forth in GenBank® Accession number AAD56538. The human HPO is an orthologue of rat augmenter of liver regeneration or hepatic stimulator substance (see, e.g., Li et al. (2000) J. Biol. Chem. 275:37443). Rat HPO is a protein of 125 amino acids (see, e.g., Hagiya et al. (1994) PNAS 91:8142). An exemplary nucleotide sequence of a partial cDNA of human HPO is set forth in SEQ ID NO: 13 and the protein encoded thereby is set forth in SEQ ID NO: 14, which sequences correspond to GenBank® Accession Numbers AF306863 and AAG38105, respectively. An exemplary partial nucleotide sequence of a mouse HPO is set forth in SEQ ID NO: 15 and the protein encoded thereby is set forth in SEQ ID NO: 16, which sequences correspond to GenBank® Accession Numbers AF148688 and AAD36987, respectively. An exemplary full length nucleotide sequence of a mouse HPO is set forth as SEQ ID NO: 17 and the protein encoded thereby is set forth in SEQ ID NO: 18. A nucleic acid encoding a protein differing in one amino acid from SEQ ID NO: 18 (the residue at position 49 is an alanine instead of a serine) is set forth in GenBank® Accession Number AB041561 and encodes the protein set forth in GenBank® Accession Number BAA95045.

“Isolated” as used herein with respect to nucleic acids, such as DNA or RNA, refers to molecules separated from other DNAs, or RNAs, respectively, that are present in the natural source of the macromolecule. For example, an isolated nucleic acid encoding an HPO polypeptide includes no more than the entire gene (including the promoter), usually no more than 10 kilobases (kb) of nucleic acid sequence which naturally immediately flanks the HPO gene in genomic DNA, preferably no more than 5 kb of such naturally occurring flanking sequences, and more preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments that are not naturally occurring as fragments and would not be found in the natural state or in a cDNA library. The term “isolated” is also used herein to refer to polypeptides that are isolated from other cellular proteins and is meant to encompass both purified and recombinant polypeptides.

The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to. The term “medium” also refers to material that is intended for use in a cell culture, even if it has not yet been contacted with cells. For example, a nutrient rich liquid prepared for cell culture is a medium.

“Non-human animals” include mammals such as rodents, non-human primates, ovines, bovines, equines, porcines, canines, felines, chickens, amphibians, reptiles, etc. “Nucleic acid” refers to polynucleotides such as deoxyribonucleic acid (DNA), and, where appropriate, ribonucleic acid (RNA). The term should also be understood to include analogs of either RNA or DNA made from nucleotide analogs, and, as applicable to the embodiment being described, single (sense or antisense) and double-stranded polynucleotides.

The term “oncostatin-M” or “OSM” includes the native oncostatin M from any organism, as well as functional mimics. An endogenous human oncostatin M is a 227 amino acid polypeptide with two glycosylation sites. An exemplary nucleotide sequence of human OSM is set forth in SEQ ID NO: 19 and the protein encoded thereby is set forth in SEQ ID NO: 20, which sequences correspond to GenBank® Accession Numbers BC011589 and AAH11589.1, respectively. An exemplary nucleotide sequence of a mouse OSM is set forth in SEQ ID NO: 21 and the protein encoded thereby is set forth in SEQ ID NO: 22, which sequences correspond to GenBank® Accession Numbers J04806 and AAA57265.1, respectively.

“Regulatory nucleic acid” means a DNA sequence that regulates expression of a selected DNA sequence operably linked thereto. Exemplary regulatory nucleic acids include promoters, enhancers, repressors, histone binding elements, etc.

The term “polypeptide”, and the terms “protein” (if single chain) and “peptide” which are used interchangeably herein, refers to a polymer of amino acids. Polypeptides may also include one or more modifications such as, for example, a lipid moiety, a phosphate, a sugar moiety, etc.

“Recombinant protein” refers to a polypeptide that is produced by recombinant DNA techniques, wherein generally, DNA encoding a protein is inserted into a suitable expression vector which is in turn used to transform a cell to produce the protein.

The following terms are used to describe the sequence relationships between two or more polynucleotides or polypeptides: “reference sequence”, “sequence identity”, “percentage of sequence identity”, and “substantial identity.” A “reference sequence” is the sequence that forms the basis for comparison. In the phrase “a polypeptide comprising an amino acid sequence that is 95% identical to the amino acid sequence in SEQ ID NO:1”, the reference sequence is the amino acid sequence shown in SEQ ID NO:1. The term “sequence identity” means that two polynucleotide sequences are identical (i.e., on a nucleotide-by-nucleotide basis) over the length of the reference sequence. The term “percentage of sequence identity” is calculated by comparing two optimally aligned sequences over the length of the reference sequence, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. Gaps may be introduced in calculating sequence identity. The term “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence, wherein the percentage of sequence identity is calculated by comparing the reference sequence to the polynucleotide sequence which may include deletions or additions which total 20 percent or less of the reference sequence over the window of comparison. Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fingal, bacterial, or algal extracts.

A “stem cell” is any cell that, if exposed to proper conditions, is capable of giving rise to two or more different cell types. A “multipotent stem cell” is a stem cell that, if exposed to proper conditions, is capable of giving rise to at least one cell type of two or more different organs or tissues. “Pluripotent stem cells” are pluripotent cells derived from pre-embryonic, embryonic, or fetal tissue at any time after fertilization, and have the characteristic of being capable under the right conditions of producing progeny of several different cell types. Pluripotent stem cells are capable of producing progeny that are derivatives of each of the three germinal layers: endoderm, mesoderm, and ectoderm, according to a standard art-accepted test, such as the ability to form a teratoma in a suitable host. Any cells of primate origin that are capable of producing progeny that are derivatives of all three germinal layers are included in the term “pluripotent stem cell.” Included in the definition of pluripotent stem cells are embryonic cells of various types, exemplified by human embryonic stem cells, e.g., as described by Thomson et al. (Science 282:1145, 1998); embryonic stem cells from other primates, such as Rhesus or marmoset stem cells, e.g., as described by Thomson et al. (PNAS, 92:7844 (1995); Developmental Biology, 38:133 (1998)); and human embryonic germ, e.g., as described in Shamblott et al. (PNAS, 95:13726 (1998)). A totipotent stem cell is the earliest stem cell in an organism and is capable of differentiating into any differentiated cell of the organism. A stem cell is said to give rise to another cell if, for example, the stem cell differentiates to become the other cell without undergoing cell division, or if the other cell is produced after one or more rounds of cell division and/or differentiation.

“Substantially pure” refers to an object species that is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species.

The term “treating” as used herein is intended to encompass preventing, curing, and/or ameliorating at least one symptom of a condition or disease.

Stem cell cultures are described as “undifferentiated” or “substantially undifferentiated” when a substantial proportion of stem cells and their derivatives in the population display morphological characteristics of undifferentiated cells, clearly distinguishing them from differentiated cells of embryo or adult origin. Undifferentiated stem cells are easily recognized by those skilled in the art, and typically appear in the two dimensions of a microscopic view with high nuclear/cytoplasmic ratios and prominent nucleoli. It is understood that colonies of undifferentiated cells within the population will often be surrounded by neighboring cells that are differentiated. Nevertheless, the undifferentiated colonies persist when the population is cultured or passaged under appropriate conditions, and individual undifferentiated cells constitute a substantial proportion of the cell population. Cultures that are substantially undifferentiated contain at least 20% undifferentiated stem cells, and may contain at least 40%, 60%, or 80%.

A “variant” of a factor, e.g., a growth factor, refers to naturally- or non-naturally-occurring polypeptides that have a certain homology to the factor, e.g., an amino acid sequence homology or a structural homology. An active variant is a variant that retains at least enough functional activity of the relevant factor that the active variant may be used as a replacement for the factor. Optionally, an active variant retains 25% or more of the activity of the relevant factor.

2. Culture Methods

In certain embodiments, the invention relates to novel methods for culturing stem cells to generate hepatocyte-like cells. The term “stem cells” includes multi-, pluri- and totipotent cells, e.g., embryonic stem cells and adult stem cells, obtained, e.g., from organisms, blastocysts or created by methods such as nuclear transfer and de-differentiation. The stem cells can be mammalian stem cells, e.g., human, non-human primate, ovine, bovine, porcine, sheep, canine, feline, mink, rabbit, hamster, rat and mouse stem cells.

In one embodiment, the stem cells are embryonic stem (ES) cells. Mouse ES cells were originally obtained from the inner cell mass of pre-implantation embryos, i.e., about 3.5 days old blastocysts (Evans et al. (1981) Nature 292:154-156; Bradley et al. (1984) Nature 309:255-258; Gossler et al. (1986) PNAS 83: 9065-9069; Robertson et al. (1986) Nature 322:445-448). Mouse ES cells from specific strains of mice and methods for obtaining such are described, e.g., in U.S. Pat. Nos. 5,985,659 and 6,190,910 by Kusakabe et al. Human ES cells and methods for obtaining such are described, e.g., in Thomson et al. (1998) Science 282:114; U.S. Pat. No. 6,200,806 by Thomson et al. and WO 00/27995 by Monash Univ. Non-human primate ES cells and methods for obtaining such are described, e.g., in Thomson et al. (1995) Proc. Natl. Acad. Sci. USA 92:7844 and U.S. Pat. No. 5,843,780 by Thomson et al. ES cells from domestic animals and and methods for obtaining such are described, e.g., in WO 90/03432; U.S. Pat. No. 6,107,543 by Sims et al. (bovine ES cells); ES cells from porcines and/or bovines are described, e.g., in Evans et al. (1990) Theriogenology, 33:125 (porcine and bovine ES cells); Notarianni et al. (1990) Proc. 4th World Cong. Genetics Applied to Livestock Production XIII, 58-64 (porcine and ovine ES cells); Notarianni et al. (1991) J. Reprod. Fert. Suppl. 43:255-260 (porcine and sheep ES cells); Piedrahita et al. (1988) Theriogenology, 29:286 (porcine ES cells); Anderson, G. B. (1992) Animal Biotechnology 3(1), 165-175 (livestock ES cells); Stewart, C. L. (1991) Animal Applications of Research in Mammalian Development, Cold Spring Harbor Laboratory Press, New York, pp. 267-283 (domestic animals ES cells); WO 95/16770 (ungulate ES cells) and WO 90/08188 (LIF from lifestock). ES cells from other species and and methods for obtaining such are described, e.g., in the following publications: Doetschman et al. (1988) Dev. Biol., 127:224 (hamster ES cells) and WO 93 03585 (chicken ES cells).

Embryonic stem cells of certain species are available publicly or commercially. For example, human ES cells are available from Wisconsin Alumni Research Foundation (WARF) (Madison, Wis.). Mouse ES cells are available from several companies, e.g., Jackson Laboratories (Bar Harbor, Me.), the American Type Culture Collection (ATCC, Manassas, Va.); and Eurogentech. Mouse ES cells are available from various strains of mice. Exemplary mouse ES cells that are commercially available include ES-E14TG2a from mouse strain 129/Ola (CRL-1821; ATCC); ES-D3 [D3] from mouse strain 129/Sv+c/+p (CRL-1934 and CRL-11632; ATCC); ES-D3 GL from mouse strain 129/Sv+c/+p (SCRC-1003; ATCC); ES-C57BL/6 from mouse strain C57BL/6j (SCRC-1002; ATCC); 9TR#1 from mouse strain 129/Sv+c/+p having disrupted TNF genes (CRL-11379; ATCC); and TK#1 from mouse strain 129/Sv+c/+p having disrupted IRF-2 genes (CRL-11383; ATCC).

Human embryonic stem (hES) cells can be prepared as described by Thomson et al. (U.S. Pat. No. 5,843,780; Science 282:1145, 1998; Curr. Top. Dev. Biol. 38:133 ff., and 1998; Proc. Natl. Acad. Sci. USA 92:7844, 1995). Briefly, human blastocysts are obtained from human in vivo preimplantation embryos. Alternatively, in vitro fertilized (IVF) embryos can be used, or one cell human embryos can be expanded to the blastocyst stage (Bongso et al., Hum Reprod 4: 706, 1989). Human embryos are cultured to the blastocyst stage in G1.2 and G2.2 medium (Gardner et al., Fertil. Steril. 69:84, 1998). Blastocysts that develop are selected for ES cell isolation. The zona pellucida is removed from blastocysts by brief exposure to pronase (Sigma). The inner cell masses are isolated by immunosurgery, in which blastocysts are exposed to a 1:50 dilution of rabbit anti-human spleen cell antiserum for 30 minutes, then washed for 5 minutes three times in DMEM, and exposed to a 1:5 dilution of Guinea pig complement (Gibco) for 3 min (see Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on murine endothelial fibroblast (mEF) feeder layers.

After 9 to 15 days, inner cell mass-derived outgrowths are dissociated into clumps either by exposure to calcium and magnesium-free phosphate-buffered saline (PBS) with 1 mM EDTA, by exposure to dispase or trypsin, or by mechanical dissociation with a micropipette; and then replated on mEF in fresh medium. Dissociated cells are replated on mEF feeder layers in fresh ES medium, and observed for colony formation. Colonies demonstrating undifferentiated morphology are individually selected by micropipette, mechanically dissociated into clumps, and replated. ES-like morphology is characterized as compact colonies with apparently high nucleus to cytoplasm ratio and prominent nucleoli. Resulting ES cells are then routinely split every 1-2 weeks by brief trypsinization, exposure to Dulbecco's PBS (without calcium or magnesium and with 2 mM EDTA), exposure to type IV collagenase (about 200 U/mL; Gibco) or by selection of individual colonies by micropipette. Clump sizes of about 50 to 100 cells are optimal.

Human embryonic germ (hEG) cells can be prepared from primordial germ cells present in human fetal material taken about 8-11 weeks after the last menstrual period. Suitable preparation methods are described in Shamblott et al., Proc. Natl. Acad. Sci. USA 95:13726, 1998 and U.S. Pat. No. 6,090,622. Briefly, genital ridges are rinsed with isotonic buffer, then placed into 0.1 mL 0.05% trypsin/0.53 mM sodium EDTA solution (BRL) and cut into <1 mm³ chunks. The tissue is then pipetted through a 100 μL tip to further disaggregate the cells. It is incubated at 37° C. for about.5 min, then about 3.5 mL EG growth medium is added. EG growth medium is DMEM, 4500 mg/L D-glucose, 2200 mg/L mM sodium bicarbonate; 15% ES qualified fetal calf serum (BRL); 2 mM glutamine (BRL); 1 mM sodium pyruvate (BRL); 1000-2000 U/mL human recombinant leukemia inhibitory factor (LIF, Genzyme); 1-2 ng/ml human recombinant basic fibroblast growth factor (bFGF, Genzyme); and 10 μM forskolin (in 10% DMSO). In an alternative approach, EG cells are isolated using hyaluronidase, collagenase, and DNAse. Gonadal anlagen or genital ridges with mesenteries are dissected from fetal material, the genital ridges are rinsed in PBS, then placed in 0.1 ml HCD digestion solution (0.01% hyaluronidase type V, 0.002% DNAse I, 0.1% collagenase type IV, all from Sigma prepared in EG growth medium). Tissue is minced and incubated 1 h or overnight at 37° C., resuspended in 1-3 mL of EG growth medium, and plated onto a feeder layer.

Ninety-six well tissue culture plates are prepared with a sub-confluent layer of feeder cells cultured for 3 days in modified EG growth medium free of LIF, bFGF or forskolin, inactivated with 5000 rad γ-irradiation. Suitable feeders are STO cells (ATCC Accession No. CRL 1503). About 0.2 mL of primary germ cell (PGC) suspension is added to each of the wells. The first passage is conducted after 7-10 days in EG growth medium, transferring each well to one well of a 24-well culture dish previously prepared with irradiated STO mouse fibroblasts. The cells are cultured with daily replacement of medium until cell morphology consistent with EG cells are observed, typically after 7-30 days or 1-4 passages.

Mouse ES cells can be obtained, e.g., as described in M. L. Roach and J. D. McNeish (2002) Methods in Mol. Biol. 185:1. Briefly, day 3.5 post coitus (p.c.) plugged mice females are sacrificed and the blastocyst stage embryos are flushed from uterine horns. The embryos are washed and transferred onto fresh feeder layers or in media containing about 1,000 units/ml LIF (ESGRO). When the embryos have attached to the dish, the inner cell mass (ICM) is removed from the rest of the embryo and transferred into a dish with fresh media and feeder layers and/or LIF. The next day, the ICM, which should be attached to the dish, is treated with trypsin and split. The cells are then cultured for several days during which the media is changed every day and every second or third day, the colonies are passed. The colonies should not grow larger than 400 μm in diameter. The cells are then grown in progressively larger sized dishes.

Other stem cells that can be used include embryonic germ cell lines, e.g., obtained from fetal gonadal tissue or from tissue formed after gestation. Pluripotent human embryonic cell lines derived from cultured human primordial germ cells are described, e.g., in Shamblott et al., PNAS, 95:13726 (1998) and PCT International Patent Publication No. WO 98/43679. Primordial germ cells and their isolation are also described, e.g., in U.S. Pat. Nos. 5,453,357 and 5,690,926 by Hogan et al. (mouse and non-mouse primordial germ cells); U.S. Pat. No. 6,090,622 by Gearhart and Shamblott (human pluripotential embryonic germ cells) and U.S. Pat. No. 6,194,635 by Anderson (porcine primordial germ cells).

In other embodiments, the stem cells are adult stem cells, such as liver stem cells (e.g. oval cells), mesenchymal stem cells, pancreatic stem cells, multipotent adult stem cells and other stem cells that are able to give rise to hepatocyte-like cells when cultured according to a method described herein. Exemplary stem cells and methods of isolating such are described, e.g., in U.S. Pat. No. 5,861,313 by Pang et al. (pancreatic and hepatic progenitor cells); U.S. Pat. Nos. 6,146,889; 6,069,005; and 6,242,252 by Reid et al. (hepatic progenitor cells); and PCT International Patent Publication Nos. WO 01/11011 (multipotent adult stem cell lines); as well as WO 00/43498 and WO 00/36091 (human liver progenitor cells).

Hepatocyte-like cells of this invention can be genetically altered in a manner that permits the genetic alteration to be either transient, or stable and inheritable as the cells divide. Undifferentiated cells can be genetically altered and then differentiated into the desired phenotype, or the cells can be differentiated first before genetic alteration. Where the stem cells are genetically altered before differentiation, the genetic alteration can be performed on a permanent feeder cell line that has resistance genes for drugs used to select for transformed cells, or on stem cells grown in feeder-free culture.

Suitable methods for transferring vector or plasmids into stem cells include lipid/DNA complexes, such as those described in U.S. Pat. Nos. 5,578,475; 5,627,175; 5,705,308; 5,744,335; 5,976,567; 6,020,202; and 6,051,429. Suitable reagents include lipofectamine, a 3:1 (w/w) liposome formulation of the poly-cationic lipid 2,3-dioleyloxy-N-[2(sperminecarboxamido)ethyl]-N,N-dimethyl-1-propanaminium trifluoroacetate (DOSPA) (Chemical Abstracts Registry name: N-[2-(2,5-bis[(3-aminopropyl)amino]-1-oxpentyl}amino) ethyl]-N,N-dimethyl-2,3-bis(9-octadecenyloxy)-1-propanaminium trifluoroacetate), and the neutral lipid dioleoyl phosphatidylethanolamine (DOPE) in membrane filtered water. Exemplary is the formulation Lipofectamine 2000™ (available from Gibco/Life Technologies #11668019). Other reagents include: FuGENE™ 6 Transfection Reagent (a blend of lipids in non-liposomal form and other compounds in 80% ethanol, obtainable from Roche Diagnostics Corp. #1814443); and LipoTAXI™ transfection reagent (a lipid formulation from Invitrogen Corp., #204110). Transfection of ES cells can also be performed by electroporation, e.g., as described in M. L. Roach and J. D. McNeish (2002) Methods in Mol. Biol. 185:1. Suitable viral vector systems for producing stem cells with stable genetic alterations may be based on adenoviruses and retroviruses, and may be prepared using commercially available virus components.

In certain embodiments, stem cells can be stably transfected with a marker that is under the control of a hepatocyte-specific regulatory region, such that during differentiation, the marker is selectively expressed in the hepatocyte-like cells, thereby allowing selection of the hepatocyte-like cells relative to the cells that do not express the marker. The marker can be, e.g., a cell surface protein or other detectable marker, or a marker that can make cells resistant to conditions in which they die in the absence of the marker, such as an antibiotic resistance gene. These methods are further described, e.g., in U.S. Pat. No. 6,015,671. Hepatocyte specific promoters include the promoter of late stage hepatocyte markers, e.g., as described herein. Accordingly, hepatocyte-like cells can be further purified by selection of the cells expressing such a marker, e.g., by selection on a medium that kills cells that do not express the marker (e.g., in the presence of an antibiotic if the marker is an antibiotic resistance marker), or by selecting cells that are positive for the marker by, e.g., fluorescence activated cell sorting (FACS), panning or using beads.

In certain embodiments, stem cells are exposed to one or more culture conditions in an appropriate sequence and for an appropriate time so as to generate a cell population comprising hepatocyte-like cells. In certain embodiments, one or more of the culture conditions comprises one or more of the following growth factors: aFGF, EGF, HGF, OSM, HPO, nicotinamide, dexamethasone, insulin and transferrin. Optionally, the method comprises exposing the cells to an early culture condition comprising aFGF and EGF; a middle culture condition comprising HGF; and/or a late culture condition comprising oncostatin M. The factors used in the early, middle and late conditions need not be mutually exclusive, and cells need not be exposed to these conditions in an uninterrupted succession. Cells may be frozen or otherwise stored between various steps, and cells may be exposed to intervening culture conditions, so long as the intervening culture conditions do not disrupt the program of differentiation caused by the combination of early, middle and late conditions.

In certain embodiments, the cells are exposed to an early culture condition comprising EGF and/or aFGF, a middle culture condition comprising EGF, aFGF and/or HGF, and a late culture condition comprising EGF, HGF and/or OSM.

In another embodiment, the cells are exposed to an early culture condition comprising EGF, aFGF and/or nicotinamide; a middle culture condition comprising EGF, aFGF, HGF and/or nicotinamide; and a late culture condition comprising EGF, HGF, OSM, nicotinamide, insulin, transferrin, selenium-G and/or dexamethasone.

In certain embodiments, cells are exposed to culture conditions comprising HPO, e.g., HPO is added to a middle and/or late culture condition.

The precise concentration of growth factor to be used in any one culture condition may be optimized and may vary depending on the source of growth factor and the form (e.g. purified from a natural source, produced as a recombinant form, a fragment or variant or a functional mimic). In an exemplary embodiment, EGF may be used at a concentration ranging from about 1-50 ng/ml; preferably from about 1-20 ng/ml; even more preferably from about 5-15 ng/ml; and most preferably about 10 ng/ml. In an exemplary embodiment, aFGF may be used at a concentration ranging from about 1-50 ng/ml; preferably about 1-20 ng/ml; even more preferably about 5-15 ng/ml; and most preferably about 10 ng/ml. In an exemplary embodiment, HGF may be used at a concentration ranging from about 5-100 ng/ml; more preferably about 5-50 ng/ml;even more preferably about 15-30 ng/ml; and most preferably about 25 ng/ml. In an exemplary embodiment, OSM may be used at a concentration of 1-50 ng/ml; more preferably about 1-30 ng/ml; even more preferably about 5-15 ng/ml; and most preferably about 10 ng/ml. In an exemplary embodiment, HPO may be used at a concentration ranging from about 10-250 ng/ml; more preferably about 20-100 ng/ml; even more preferably about 40 to 60 ng/ml; and most preferably about 50 ng/ml. In an exemplary embodiment, nicotinamide may be used at a concentration ranging from about 1-50 μM more preferably about 1-30 μM; even more preferably about 5-15 μM; and most preferably about 10 μM. In an exemplary embodiment, dexamethasone may be used at a concentration of 20-500 nM; preferably about 20-200 nM; even more preferably about 80-120 nM; and most preferably about 100 nM. In an exemplary embodiment, insulin may be used at a concentration of about 0.1 -100 μg/ml; preferably about 1-50 μg/ml; even more preferably about 5-20 μg/ml; and most preferably about 10 μg/ml. In an exemplary embodiment, transferrin may be used at a concentration of 0.1-100 μg/ml; preferably about 1-50 μg/ml; even more preferably about 1-10 μg/ml; and most preferably about 5 μg/ml. Transferrin in HepEB medium is preferably present at a concentration of about 10 to about 1000 μg/ml; more preferably about 100 to about 1000 μg/ml; even more preferably about 200 to about 500 μg/ml; and most preferably about 300 μg/ml. In an exemplary embodiment, selenium may be used at a concentration of 0.1-100 ng/ml; preferably about 1-50 ng/ml; even more preferably about 1-10 ng/ml; and most preferably about 5 ng/ml.

Optionally, polypeptide growth factors are matched to the species of the cells. For example, it may be desirable to use human EGF when working with human cells and murine EGF when working with murine cells.

In addition to the appropriate growth factors, other media components may be selected as appropriate for the cellular starting material, and some degree of routine optimization is expected for each culture situation. For example, commonly used media bases include Dulbecco's Modified Eagle's Medium (DMEM), Ham's F-12 nutrient mixture, Iscove's Modified Dulbecco's Medium (IMDM), McCoy's 5A, RPMI 1640, etc. Generally, differences between the different media can be compensated for with the addition or omission of supplements, such as carbon sources (e.g. glucose, pyruvate, etc.), serum (e.g. fetal bovine serum), vitamins, amino acids, etc. Other media components that may be selected and optimized to match the desired culture conditions are antibiotics (e.g. aminoglycosides such as gentamycin, penicillins, etc.) amino acids (particularly glutamine) and reducing agents (e.g. thiols such as monothioglycerol).

In certain embodiments, the cells are cultured with one or more of the Hep EB, Hep I, Hep II, Hep III, Hep IV media described in the Examples below or variants thereof.

Each of the differentiation steps described herein can be conducted for a time appropriate to get the cells ready for the next differentiation step. Generally, each differentiation step takes from 2-4 days, preferably 3 days.

In a particular embodiment, differentiation of ES cells is conducted as follows. ES cells cultured in the presence of a feeder layer and/or leukemia inhibitory factor (LIF) are removed from the feeder layer and/or LIF, such as to allow differentiation. During this first stage, referred to as the “embryoid body stage,” ES cells form embryoid bodies. This first stage extends from day 0 to about day 5, with day 0 corresponding to the day the feeder layer and/or LIF is removed, such that differentiation may begin. The cells may be cultured in media, e.g., containing transferrin, e.g., the HepEB media described in the Examples. In a second stage, referred to as the “early stage,” consisting of about day 6 to day 8, the embryoid bodies are cultured in a medium comprising EGF, aFGF, and optionally nicotinamide. For example, the cells can be cultured in the medium HepI described in the Examples. Towards the end of this stage, e.g., day 8, the embryoid bodies may be very spread and may be touching each other. At this point, the embryoid bodies are dissociated into single cells and cultured in the same medium as prior to the dissociation. In a third stage, referred to as the “middle stage,” consisting of about day 9 to day 11, the cells are cultured in a medium comprising HGF, and optionally EGF, aFGF, and/or nicotinamide. For example, the cells can be cultured in the medium HepII described in the Examples. During this stage, the cells form a nice monolayer that is about 60-70% confluent. Cells may be passed during this stage, e.g., on day 11. In a fourth stage, referred to as the “late stage,” consisting of about day 12-14, the cells are cultured in a medium comprising OSM, and optionally EGF, HGF, dexamethasone, insulin, transferrin, and/or selenium-G. For example, the cells can be cultured in the medium HepIII described in the Examples. During this stage, the cells appear flatter, more epithelial-like in morphology and 60-70% confluent. The cells may be passed during this stage, e.g., on day 14.

During the various culture steps of the ES cells and derivatives thereof, when the cells are not cultured in the presence of feeder layers and/or LIF, the cells may be cultured on dishes coated with collagen, e.g., collagen type I. For example, cell can be cultured on coated dishes from the moment they start forming embryoid bodies. In one embodiment, the cells are cultured on non-coated dishes until about day 5, at which point the cells which are in the form of embryoid bodies are transferred to collagen type I coated dishes. Other coatings that may be used include fibronectin, e.g., 0.1 μg/ml, and Matrigel (containing a mixture of extracellular matrix (ECM) components), e.g., 1% Matrigel.

Differentiation of ES cells can also be promoted by withdrawing serum or serum replacement from medium, withdrawing a factor that promotes proliferation, withdrawing a factor that inhibits differentiation, or adding a new factor that promotes differentiation.

When cells which are not ES cells are used for differentiation into hepatocyte-like cells, the first stage of differentiation may correspond to the second or third stage of differentiation of ES cells, i.e., the early or middle stage, respectively. The cells may then be taken through the later steps of differentiation described above for the ES cells.

The differentiation of cells can be monitored by visual inspection of the cells. The differentiation can also be monitored by analysis of phenotypic or functional characteristics of ES cells, hepatocytes and precursors thereof. For example, differentiation can be monitored by analysis of expression of early and late markers of hepatocyte differentiation. Exemplary early markers include hepatocyte nuclear factor (HNF)-3β, GATA4, CK19 and α-fetoprotein, as described, e.g., in Schwartz et al. (2002) J. Clin. Invest. 109: 1291. Late markers of hepatocyte differentiation include CK18, albumin and HNF-1α (see, e.g., Schwartz et al., supra). Other tests that can be used are further described herein, in particular, in the Examples.

In another embodiment, the invention relates to methods for selecting hepatocyte-like cells from a population of cells, e.g., a population of cells obtained from the differentiation of stem cells as described above. In one embodiment, a cell population comprising, or suspected of comprising, a hepatocyte-like cell is placed in a culture condition that favors the growth or survival of hepatocytes, e.g., by selecting for gluconeogenic cells over non-gluconeogenic cells. It is generally accepted that only two types of cells in the human body are capable of performing gluconeogenesis: hepatocytes and mammary gland epithelial cells. Accordingly, a culture condition that favors the growth or survival of gluconeogenic cells will tend to enrich (or select) for hepatocytes versus essentially all other cell types. Conditions that favor growth or survival of gluconeogenic cells include, for example, conditions where the most significant, or, optionally, the sole carbon source is a carbon source that supports the growth of gluconeogenic cells but not non-gluconeogenic cells. An exemplary carbon source of this type is pyruvate. Accordingly, a medium containing reduced amounts of glucose, or no glucose at all, and pyruvate will tend to favor the growth or survival of gluconeogenic cells. Compounds that can be converted to pyruvate may also be used. An exemplary medium for selecting hepatocyte-like cells comprises sufficient pyruvate to permit survival or growth of gluconeogenic cells and contains insufficient nutrients (e.g. glucose) to support the survival or growth of non-gluconeogenic cells. Exemplary pyruvate concentrations range from about 0.1-30 mM; more preferably about 0.1-10 mM; even more preferably about 0.2-5 mM; and most preferably about 1 mM. In certain embodiments pyruvate is supplied as pyruvic acid.

In an exemplary embodiment, a stem cell is differentiated into a hepatocyte-like cell as described above, e.g., by culture through stages 1-4 described above, and then subjected to the enrichment step described above, which is also referred to as a “maturation and selection stage” (stage 5). For example, ES cells subjected to stages 1-4 of differentiation can then be subjected to the maturation and selection stage (stage 5), consisting of about day 15-18. During this stage, the cells are cultured in a medium that is selective for gluconeogenic cells, as described herein and known to those of skill in the art. For example, the cells can be cultured in a medium comprising pyruvic acid or pyruvate and optionally one or more of EGF, HGF, OSM, dexamethasone, and/or sodium butyric acid. In a preferred embodiment, the cells can be cultured in the medium HepIV described in the Examples. Towards the end of this stage, a lot of cell death will be observed. The medium can be removed and the cells washed every other day or every day. On day 19 or 20, the cells can be washed and further incubated in a glucose containing medium, comprising, e.g., EGF, HGF, and/or OSM, such as the medium HepIII described in the Examples.

In a further embodiment, butyrate is employed as an agent that favors the retention of hepatocyte-like cells, as evidenced by the retention of hepatocyte characteristics. Exemplary media comprise butyrate at concentrations ranging from 0.1 mM to 25 mM; preferably about 1-15 mM; more preferably about 2-10 mM; and most preferably about 5 mM. In certain embodiments butyrate is provided as sodium butyrate. Dimethylsulfoxide (DMSO) may be used in a similar manner. For example, a medium may comprise 2-50 mg/ml DMSO; preferably about 5-30mg/ml; more preferably about 5-20mg/ml and most preferably about 10 mg/ml.

In an additional embodiment, a cell population comprising, or suspected of comprising, hepatocyte-like cells is exposed to a gluconeogenic medium comprising butyrate or DMSO. Optionally, the medium comprises pyruvate and butyrate or DMSO at the ranges of concentrations described above. HEP IV, described below in the examples, is an exemplary medium of this type.

In yet a further embodiment, methods of the invention may include exposing cells to a culture condition that is suitable for activation of hepatocyte-like cells, or, in other words, increasing the level of one or more hepatocyte metabolic activities. For example, phenobarbital and chemically related compounds are known to induce the expression of one or more cytochrome P450 enzymes in hepatocytes, particularly in human hepatocytes. Pyrethroids (e.g. permethrin, cypermethrin, and fenvalerate) may be used in a similar manner. Heder et al. Biochem Pharmacol 2001 62(1):71-9. Pregnenolones, such as pregnenolone 16α-carbonitrile also induce the expression or activity of various cytochrome P450 enzymes, particularly in rodent cells. Dexamethasone may be used similarly. In certain embodiments, pregnenolone 16α-carbonitrile is used at a concentration ranging from 10 nM to 1 mM; preferably about 50-500 nM; more preferably about 90-200 nM; and most preferably about 100 nM. For example, cells can be subjected to this stage (stage 6) by incubation in a medium, e.g., HepIII, comprising one such agent. After one day of incubation, the medium of the cells can be replaced by a medium comprising one or more of OSM, nicotinamide, dexamethasone, insulin, transferrin, and selenium. An examplary medium is medium HepV described in the Examples.

In general, cells are exposed to a condition that activates hepatocyte-like cells at a stage when an enriched population of hepatocyte-like cells has been obtained.

Certain methods described herein employ polypeptide growth factors. Preparations of each of these factors are commercially available (with the exception of HPO), and sources from which they can be obtained are provided in the Examples. It is also understood that one may produce these factors according to methods known in the art. Exemplary nucleotide and amino acid sequences for these factors are provided in the attached sequence listing and are further described herein. For simplicity, the nucleotide and amino acid sequences of the various growth factors, described herein, and corresponding GenBanks Accession Numbers, if any, have the following SEQ ID NOs:

TABLE 1
SEQ ID NOs of factors described herein

| | | GenBank ® Accession |
|---|---|---|
| Sequence | SEQ ID NO | Number (if any) |

| | | |
|---|---|---|
| Human aFGF nucleotide sequence | SEQ ID NO: 1 | NM_000800 |
| Human aFGF amino acid sequence | SEQ ID NO: 2 | NP_000791.1 |
| Mouse aFGF nucleotide sequence | SEQ ID NO: 3 | M30641 |
| Mouse aFGF amino acid sequence | SEQ ID NO: 4 | AAA37618.1 |
| Human EGF nucleotide sequence | SEQ ID NO: 5 | NM_001963 |
| Human EGF amino acid sequence | SEQ ID NO: 6 | NP_001954.1 |
| Mouse EGF nucleotide sequence | SEQ ID NO: 7 | J00380 |
| Mouse EGF amino acid sequence | SEQ ID NO: 8 | AAA37539.1 |
| Human HGF nucleotide sequence | SEQ ID NO: 9 | M29145 |
| Human HGF amino acid sequence | SEQ ID NO: 10 | AAA52650.1 |
| Mouse HGF nucleotide sequence | SEQ ID NO: 11 | D10212 |
| Mouse HGF amino acid sequence | SEQ ID NO: 12 | BAA01064.1 |
| Human partial HPO nucleotide sequence | SEQ ID NO: 13 | AF306863 |
| Human partial HPO amino acid sequence | SEQ ID NO: 14 | AAG38105 |
| Mouse partial HPO nucleotide sequence | SEQ ID NO: 15 | AF148688 |
| Mouse partial HPO amino acid sequence | SEQ ID NO: 16 | AAD36987 |
| Mouse full length HPO nucleotide sequence | SEQ ID NO: 17 | see Sequence Listing |
| Mouse full length HPO amino acid sequence | SEQ ID NO: 18 | see Sequence Listing |
| Human OSM nucleotide sequence | SEQ ID NO: 19 | BC011589 |
| Human OSM amino acid sequence | SEQ ID NO: 20 | AAH11589.1 |
| Mouse OSM nucleotide sequence | SEQ ID NO: 21 | J04806 |
| Mouse OSM amino acid sequence | SEQ ID NO: 22 | AAA57265.1 |

Regarding HPO, the short form or the full length form may be used in differentiation. A nucleic acid encoding a human homolog of the full length form of HPO can be isolated, e.g., by PCR using primers based on the sequence set forth in AF306863, AF183892 and SEQ ID NO: 17.

The factors are encoded as a precursor protein, a portion of which becomes the mature factor. The location of the signal peptide for each of these is known in the art. A person of skill in the art will readily recognize that variants, fragments, functional mimics and orthologs can be used, provided that such compounds can be provided at a concentration sufficient to provide similar functional activity.

For example, a variant may be generated by making conservative amino acid changes and testing the resulting variant in one of the functional assays described above or another functional assay known in the art. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

As those skilled in the art will appreciate, variants or fragments of polypeptide growth factors can be generated using conventional techniques, such as mutagenesis, including creating discrete point mutation(s), or by truncation. For instance, mutation can give rise to variants which retain substantially the same, or merely a subset, of the biological activity of a polypeptide growth factor from which it was derived.

Growth factor variants may also be chemically modified by forming covalent or aggregate conjugates with other chemical moieties, such as glycosyl groups, lipids, phosphate, acetyl groups and the like. Covalent derivatives can be prepared by linking the chemical moieties to functional groups on amino acid sidechains of the protein or at the N-terminus or at the C-terminus of the polypeptide.

Functional mimics of a growth factor include any compound that has an effect on at least a portion of the cellular signaling pathway of the relevant growth factor and is able to elicit a similar response in a functional assay for the growth factor, such as in one of the assays disclosed herein. As with fragments and variants, a functional mimic need not have the same concentration range for effectiveness, so long as the functional mimic is sufficiently active and non-toxic that there exists a practical concentration at which it can be used. A functional mimic may be generated by, for example, designing a molecule that activates the growth factor receptor, i.e., an EGF functional mimic could be a molecule that activates the EGF receptor.

For further elaboration of general techniques useful in the practice of this invention, the practitioner can refer to standard textbooks and reviews in cell biology, tissue culture, and embryology. Included are Teratocarcinomas and Embryonic Stem Cells: A Practical Approach (E. J. Robertson, ed., IRL Press Ltd. 1987); Guide to Techniques in Mouse Development (P. M. Wasserman et al., eds., Academic Press 1993); Embryonic Stem Cell Differentiation In Vitro (M. V. Wiles, Meth. Enzymol. 225:900, 1993); Properties and uses of Embryonic Stem Cells: Prospects for Application to Human Biology and Gene Therapy (P. D. Rathjen et al., al.,1993). Differentiation of stem cells is reviewed in Robertson, Meth. Cell Biol., 75:173 (1997); as well as Pedersen, Reprod. Fertil. Dev., 10:31 (1998).

Proteins can be produced, e.g., by expression of a nucleic acid encoding the protein in a eukaryotic or prokaryotic system or in an in vitro translation system according to techniques well known in the art. It is preferable to express a protein in a eukaryotic system, such that the protein has the proper posttranslational modifications.

Human HPO can be produced recombinantly as described, e.g., in Yang et al., Acta Biochim. Biophys. Sin., 29:414 (1997).

EGF activity may be tested by measuring the ability of a compound to stimulate ³H-thymidine incorporation in an EGF-responsive mouse fibroblast cell line, such as the Balb/3T3 cell line. Rubin et al., PNAS, 88:415 (1991). In this type of assay, human recombinant EGF will have an ED₅₀ typically in the range of 0.1-0.4 ng/ml. An EGF variant, fragment or functional mimic need not have a similar ED₅₀, but the activity should be sufficiently high that the compound can be used in a culture medium at a reasonable concentration. A functional mimic for EGF may be, for example, a compound that is an agonist for an EGF receptor.

HGF activity may be tested by measuring the ability of a compound to stimulate ³H-thymidine incorporation in the HGF-responsive monkey epithelial cell line, 4MBr-5. Rubin et al., PNAS, 88:415 (1991). In this type of assay, human recombinant HGF will have an ED₅₀ typically in the range of 20-40 ng/ml. An HGF variant, fragment or functional mimic need not have a similar ED₅₀, but the activity should be sufficiently high that the compound can be used in a culture medium at a reasonable concentration. A functional mimic for HGF may be, for example, a compound that is an agonist for an HGF receptor.

OSM activity may be tested by measuring the ability of a compound to stimulate ³H-thymidine incorporation in quiescent NIH/3T3 cells. In this type of assay, mouse recombinant OSM will have an ED₅₀ typically in the range of 2-4 ng/ml. Human OSM activity may also be tested by measuring proliferation of a factor-dependent human erythroleukemic cell line, TF-1. Kitamura et al., J. Cell Physiol., 140:323-34 (1989). In this type of assay, human recombinant OSM will have an ED₅₀ typically in the range of 0.1-3 ng/ml. An OSM variant, fragment or functional mimic need not have a similar ED₅₀, but the activity should be sufficiently high that the compound can be used in a culture medium at a reasonable concentration. A functional mimic for OSM may be, for example, a compound that is an agonist for an OSM receptor.

aFGF activity may be tested by measuring the ability of a compound to stimulate ³H-thymidine incorporation in an aFGF-responsive mouse fibroblast cell line, such as the Balb/3T3 cell line. Rubin et al., PNAS, 88:415 (1991). In this type of assay, human recombinant aFGF will have an ED₅₀ typically in the range of 2-10 ng/ml. An aFGF variant, fragment or functional mimic need not have a similar ED₅₀, but the activity should be sufficiently high that the compound can be used in a culture medium at a reasonable concentration. A functional mimic for aFGF may be, for example, a compound that is an agonist for an aFGF receptor.

HPO activity may be tested by measuring the ability of a compound to stimulate ³H-thymidine incorporation in an HPO-responsive hepatocyte cell line, such as the HepG2 cell line, as described, e.g., in Wang et al., J. Biol. Chem., 274:11469 (1999). An HPO variant, fragment or functional mimic should have an activity sufficiently high that the compound can be used in a culture medium at a reasonable concentration. A functional mimic for HPO may be, for example, a compound that is an agonist for an HPO receptor, which is described, e.g., in Wang et al., supra.

3. Hepatocyte-like Cells

The invention provides enriched populations of hepatocyte-like cells. Exemplary populations of cells comprise at least about 50%; preferably at least about 60%; 70%; 80%; 90%; 95%; 98% and most preferably 99% of hepatocyte-like cells. As set forth in the Examples, the methods described herein, e.g., differentiation through stages 1-4, permit the obtention of a population of cells in which at least about 50% of the cells are hepatocyte-like cells. When a maturation and selection step was added, populations of at least about 90% of hepatocyte-like cells were obtained.

Hepatocyte-like cells can be characterized as follows. The cells may also be positive for late stage markers of hepatocytes, such as HNF-1α, cytokeratin (CK) 18 and albumin; the absence of early hepatocyte markers, e.g., HNF-3β, GATA4, CK19, α-fetoprotein; express cytochrome P450 genes, e.g., CYP1A1, CYP2B1, CYP2C6, CYP2C11, CYP2C13, CYP3A2 and CYP4A1; and acquire a polarized structure. Hepatocyte-like progenitor cells may be detected by the presence of early hepatocyte markers. Other markers of interest for liver cells include α1-antitrypsin, glucose-6-phosphatase, transferrin, asialoglycoprotein receptor (ASGR), CK7, γ-glutamyl transferase; HNF 1β, HNF 3α, HNF-4α, transthyretin, CFTR, apoE, glucokinase, insulin growth factors (IGF) 1 and 2, IGF-1 receptor, insulin receptor, leptin, apoAII, apoB, apoCIII, apoCII, aldolase B, phenylalanine hydroxylase, L-type fatty acid binding protein, transferrin, retinol binding protein, and erythropoietin (EPO).

Tissue-specific markers can be detected by immunological techniques, such as flow immunocytochemistry for cell-surface markers, immunohistochemistry (for example, of fixed cells or tissue sections) for intracellular or cell-surface markers, Western blot analysis of cellular extracts, and enzyme-linked immunoassay, for cellular extracts or products secreted into the medium. The expression of tissue-specific gene products can also be detected at the MRNA level by Northern blot analysis, dot-blot hybridization analysis, or by reverse transcriptase initiated polymerase chain reaction (RT-PCR) using sequence-specific primers in standard amplification methods. Sequence data for the particular markers listed in this disclosure can be obtained from public databases such as GenBank® (URL www.ncbi.nlm.nih.gov:80/entrez). Primers for amplifying sequences of marker of interest can also be found, e.g., in Schwartz et al. (2002) J. Clin. Invest. 109:1291. Expression of tissue-specific markers as detected at the protein or mRNA level is considered positive if the level is at least 2-fold, and preferably more than 10- or 50-fold above that of a control cell, such as an undifferentiated stem cell, a fibroblast, or other unrelated cell type.

Hepatocyte-like cells may also display the following biological activities, as evidenced by functional assays. The cells may have a positive response to dibenzylfluorescein (DBF) (see Examples); have the ability to metabolize certain drugs, e.g., dextromethorphan and coumarin; have drug efflux pump activities (e.g., P glycoprotein activity); upregulation of CYP activity by phenobarbital, as measured, e.g., with the pentoxyresorufin (PROD) assay, which is seen only in hepatocytes and not in other cells (see, e.g., Schwartz et al., J. Clin. Invest., 109:1291 (2002)); take up LDL, e.g., Dil-acil-LDL (see, e.g., Schwartz et al., supra); store glycogen, as determined, e.g., by using a periodic acid-Schiff (PAS) staining of the cells (see, e.g., Schwartz et al., supra); produce urea and albumin (see, e.g., Schwartz et al., supra); and present evidence of glucose-6-phosphatase activity.

Hepatocyte-like cells may be characterized for drug efflux pump activity (e.g., P glycoprotein activity) by measuring the accumulation of various test compounds in cells that have been treated or not treated with an inhibitor of P-glycoprotein. Cells that have P-glycoprotein activity are expected to show greater cellular accumulation of the test compound in the absence of the P-glycoprotein inhibitor than in the presence of the inhibitor.

Diazepam and 7-EC metabolic activity can be measured as follows. 4×10⁶ hepatocyte-like cells are cultured in a monolayer in 5 ml of medium containing 50 μg/ml diazepam or 7-EC and the amount of diazepam or 7-hydroxycoumarin metabolites present in the culture supernatant measured after 3 hours of culture, respectively. Diazepam and 7-hydroxycoumarin metabolites can be assayed by high performance liquid chromatography (HPLC) using a C18 μ-Bondpack reverse phase column according to known methods, e.g., Jauregui et al., Xenobiotica, 21:1091-106 (1991).

Acetaminophen and its metabolites can be determined by ion-pairing HPLC using a C18 reverse phase column. Acetaminophen metabolism can be measured as follows. 4×10⁶ hepatocyte-like cells are cultured in a monolayer in 5 ml of medium containing 5 mM acetaminophen (0.756 mg/ml), and the amount of acetaminophen glucuronide present in the culture supernatant measured after 3 hours of culture. The amount of acetaminophen and its metabolites, e.g., acetaminophen glucuronide, can be determined by ion-pairing high performance liquid chromatography, e.g, using the method of Colin et al., J. Chromatogr., 377:243-51 (1986). Acetaminophen may also be metabolized via a sulfonation pathway and metabolites may be assayed using methods known in the art.

Lidocaine metabolism can be measured using known methods, e.g., Jauregui et al., Hepatology, 21:460-69 (1995). For example, 4×10⁶ hepatocyte-like cells are cultured in a monolayer in 5 ml of medium containing 20 μg/ml lidocaine, and the amount of lidocaine metabolite, e.g., monoethylglycinexylidide (MEGX), present in the culture supernatant is measured after 3 hours of culture. Metabolism of lidocaine can be tested using a TDX Analyzer manufactured by Abbott Diagnostics Laboratories, No. Chicago, Ill.

Ammonia metabolism can be measured according to methods known in the art, e.g., using the commercial analyzer, Ektachem, manufactured by Kodak Corp. Rochester, N.Y. Ammonia metabolism can be detected by measuring the amount of ammonia remaining in the culture supernatant after 3 hours of culture.

4. Uses for Purified Hepatocyte Preparations

In one embodiment, hepatocyte-like cells are used for testing whether test compounds (or agents) have a biological effect, e.g., a cytotoxic effect, on hepatocytes. For example, a hepatocyte-like cell preparation is incubated in the presence or absence of a test compound for a time sufficient to determine whether the compound may have a biological effect on the cells, preferably under physiological conditions, and determining whether the test compound had a biological effect on the cells, relative to the cells that were not treated with the test compound. Cells can be incubated with various concentrations of a test compound. In an illustrative embodiment, cells are plated in the wells of a multi-well plate to which different concentrations of the test compound are added, e.g., 0 μM; 0.01 μM; 0.1 μM; 1 μM; 10 μM; 100 μM; 1 mM; 10 mM and 100 mM. Cells can be incubated for various times, e.g., 1 minute, 10 minutes, 1 hour, 2 hours, 5 hours, 10 hours, 24 hours, 36 hours or more.

The biological effect that is measured can be triggering of cell death (i.e., cytotoxicity or hepatotoxicity); a cytostatic effect; or a transforming effect on the cell, as determined, e.g., by an effect on the genotype or phenotype of the cells. The cytotoxicity on cells can be determined, e.g., by incubating the cells with a vital stain, such as trypan blue.

Such screening assays can easily be adapted to high throughput screening assays.

Hepatocyte-like cells can also be used for metabolic profiling. In one embodiment, cells or a fraction thereof, e.g., a microsome fraction, are contacted with a test agent, potentially at different concentrations and for different times, the media is collected and analyzed to detect metabolized forms of the test agent. Optionally, a control molecule, such as bufuralol is also used. Metabolic profiling can be used, e.g., to determine whether a subject metabolizes a particular drug and if so, how the drug is metabolized. For such assays, it is preferable that the hepatocyte-like cells used derive from the subject.

The hepatocyte-like cells of this invention may also be used to screen candidate compounds or environmental conditions that, e.g., affect differentiation or metabolism of the cells. The hepatocyte-like cells may further be used to obtain cell specific antibody preparations and cell-specific cDNA libraries, e.g., to study patterns of gene expression, or as an active ingredient in a pharmaceutical preparation.

In another embodiment, hepatocyte-like cells are administered to a subject in need thereof. The cells can be administered to the liver of the subject, e.g., for tissue reconstitution or regeneration. The cells may be administered in a manner that permits them to graft to the intended tissue site and reconstitute or regenerate the functionally deficient area. Prior to administration, the cells may be modified to suppress an immune reaction from the subject to the cells or vice-versa (graft versus host disease), according to methods known in the art.

Hepatocyte-like cells may be administered to a subject having a complete or partial liver failure, such as resulting from a hepatitis C infection.

Hepatocytes-like cells can be assessed in animal models for ability to repair liver damage. One such example is damage caused by intraperitoneal injection of D-galactosamine (Dabeva et al., Am. J. Pathol., 143:1606 (1993)). Efficacy of treatment can be determined by immunocytochemical staining for liver cell markers, microscopic determination of whether canalicular structures form in growing tissue, and the ability of the treatment to restore synthesis of liver-specific proteins.

Cell compositions for administration to a subject in accordance with the present invention thus may be formulated in any conventional manner using one or more physiologically acceptable carriers comprising excipients and auxiliaries which facilitate processing of the compounds into preparations which can be used pharmaceutically. Proper formulation is dependent upon the route of administration chosen. Hepatocyte-like cells can be used in therapy by direct administration, or as part of a bioassist device that provides temporary liver function while the subject's liver tissue regenerates itself following fulminant hepatic failure. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. The compositions may be packaged with written instructions for use of the cells in tissue regeneration, or restoring a therapeutically important metabolic function.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al., U.S. Pat. No. 4,683,195; Nucleic Acid Hybridization(B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXAMPLES

The present invention is further illustrated by the following examples which should not be construed as limiting in any way.

### Example 1

### Maintenance of Embryonic Stem Cells

This example describes methods used for thawing, feeding, subculturing, and freezing ES cells, as well as removing ES cells from feeder cell layers.

ES cells were thawed as follows. First a 100 mm plate with feeder cells (“feeder plate”) was prepared as follows. The feeder cells used were primary embryonic fibroblasts (PEF) cells prepared as described in “Manipulating the mouse embryo” by Brigid Hogan, Frank Costantini and Elizabeth Lacy, Cold Spring Harbor Laboratory 1986. Several days prior to thawing ES cells, feeder cells were plated onto 100 mm dishes as described in E. J. Robertson “Embryo-derived Stem Cell Lines, in Teratocarcinomas and Embryonic Stem Cells: A Practical Approach, E. J. Robertson, editor, IRL Press, Washington D.C., 1987. The day of thawing of ES cells, the media of the feeder plates was removed, the feeder cells were washed with 10 ml PBS, and 15 ml of the following media was added to each plate: stem cell media SCML consisting of KO-DMEM (Gibco/Invitrogen) to which the following ingredients were added: 15% FBS (Gibco/Invitrogen); 0.2 mM L-Glutamine (Gibco/Invitrogen); 0.1 mM MEM nonessential amino acids (Gibco/Invitrogen); 0.1 mM 2-Mercaptoethanol (Sigma); 1000 units/ml ESGRO (also known as Leukemia Inhibitory Factor or LIF) (Chemicon); and either 50 units/ml penicillin and 50 μg/ml streptomycin or 20 ng/ml gentamycin (all from Gibco/Invitrogen).

Prior to using a feeder plate, it was determined whether the feeder cells were healthy. Primary embryonic fibroblast feeders usually last about 7-10 days. The prepared feeder was placed back into the incubator to equilibrate.

A vial of ES cells containing enough cells to plate one 100 mm dish with an even spread of colonies (approximately 2-3×10⁶ cells) was removed from −150° C., plunged into a 37° C. water-bath, and the vial was agitated until the frozen suspension became a slurry. The vial was doused with alcohol and transferred to a tissue culture hood. The cell suspension was transferred from the vial to the prepared feeder plate. The plate was swirled to evenly distribute the ES cells over the entire feeder surface, and returned to the incubator.

The next morning, the media was removed and replaced with fresh SCML. The dish was returned to the incubator and cultured another day. If the cells recovered easily from the freeze/thaw, they were generally ready to be split 48 hours after thawing.

ES cell cultures were daily fed as follows. The dishes were examined for the condition of the ES cell colonies and observations were recorded. Colony morphology was monitored as a gauge of culture conditions. Healthy ES cell colonies tended to have smooth borders, and the cells were tightly packed together so the individual cells were not detectable, and the entire colony has depth so as to give a refractile ring around it. Media was removed from the healthy cells and replaced with SCML.

ES cells were subcultured as follows. The plates were fed approximately 1-2 hours prior to passage. Media was removed and replaced with fresh SCML and the dish was returned to the incubator. The cells in the dish were examined for colony morphology, density and size. As a guideline, an even distribution of colonies over the entire dish, averaging 200-400 μm in diameter and spaced 200-400 μm apart, was split 1:8. The ratio for splitting the cells was calculated, generally so as to plate 1.5-2×10⁶ cells per 100 mm dish.

Media was removed and cells were washed by adding 10 ml PBS, taking care not to disturb the cell layer. The dish was gently swirled and the PBS was immediately removed. PBS was Ca⁺⁺ and Mg⁺⁺ free, which means it will dissociate the cells if left on too long. 2 ml 0.04% Trypsin EDTA was added (for 100 mm dish; 0.5 ml per well of 6-well dish; 4 drops per well of 24-well dish) to the center of the dish and the dish was rotated to distribute the trypsin over the cell layer. Cells were incubated for 1-2 min. The dish was then tapped to dislodge the cells. Longer incubations were used if cells did not float free. Generally cells were not exposed to trypsin for more than a couple of minutes, as the trypsin tends to cause cell lysis if left on too long.

A new feeder was prepared by removing old media, washing with PBS as described above and then adding 15 ml SCML. Each time the cells were exposed to trypsin was considered a passage.

Once the cells were no longer attached, 8 ml SCML was added to the trypsin cell suspension and pipetted up and down vigorously to dissociate the cells (addition of the SCML inactivated the trypsin). 1 ml cell suspension was transferred into each previously prepared feeder dishes.

Freezing of ES cells was conducted as follows. The ES cell suspension described above obtained after trypsinization was transferred into a 15 ml tube, and the cells were pelleted by centrifugation at 1000 rpm for 5 min. The supernatant was removed, taking care not to disturb the pellet. Usually a 100 mm dish yielded enough cells to freeze 4 vials (approximately 2-3×10⁶ cells/vial). 1 ml of freezing medium (50% FBS; 10% DMSO; 40% SCML)/vial was added and pipetted up and down to obtain a single cell suspension. 1 ml cell suspension was placed in each cryovial, and cap tighted to obtain a tight seal. Freezing data was recorded in a data book. Cryovials were initially frozen in a −80° C. freezer. The next day vials of frozen cells were transferred to a −150° C. freezer for long-term storage.

ES cells were removed from feeder layers as follows. When cells were ready to be split, old media was removed and cells were washed with PBS, taking care not to disturb the colonies. The PBS wash was removed and 2 ml trypsin was added (the trypsin was fresh and warm). The dish was immediately examined under a microscope. The dish was tapped during examination at 100× to dislodge the colonies. Only colonies with the correct ES cell morphology tended to come off easily. When the feeder layer started to pull loose, the dish was taken to a sterile hood. This process generally took about 30 seconds. In the hood, the dish was tilted so the trypsin and colonies pooled at the lowest point, and 8 ml SCML was added down the slope of the dish, followed by aspiration of the entire colony suspension back into the pipette.

The colony was transferred into a 15 ml tube and pipetted up and down to dissociate the colonies into smaller clumps. Cells were pelleted by centrifugation at 1000 RPM for 5 minutes. The supernatant was aspirated and the cells were resuspended in 15 ml SCML and plated in a tissue culture treated dish without feeders. For cells that are not DBA-252 ES cells it is best to use gelatin coated dishes. ES cells from other mouse strains do not attach to plastic very well and work better with the matrix gelatin.

From this point on the cells were considered to be without feeders and after another passage were ready for in vitro differentiation. The standard maintenance protocol (above) was followed, but without feeders.

These methods are also described in M. Roach and J. McNeish (2002) Methods Mol. Biol. 185:1.

### Example 2

### Generation of Hepatocyte-like Cells from Embryonic Stem Cells

This example describes the differentiation of mouse ES cells into hepatocyte-like cells. The differentiation is described as comprising several steps: an embryoid body stage (days 0-5) during which embryoid bodies are formed; an early stage (days 6-8) during which the embryoid bodies were dissociated into a single cell suspension; a middle stage (days 9-11) during which cells formed a monolayer about 60-70% confluent; and a late stage (days 12-14) during which the cells were generally flatter, more epithelial-like in morphology and 60-70% confluent. Although a highly enriched population of hepatocyte-like cells was obtained at this stage, the differentiated cells were taken through a maturation and selection stage (days 15-18) during which non-hepatocyte-like cells are killed.

The cells were cultured as follows during the embryoid body stage (day 0-5). ES DBA252 cells were used in this Example. The preparation of these cells is described in Roach et al. (1995) Exp. Cell Res. 221:520. The ES cells used for in vitro differentiation were grown without primary embryonic fibroblast (PEF) feeders in the stem cell media (SCML) that contains 1000 u/ml of leukemia inhibitory factor (LIF). When feeding feeder free ES cells care was taken so that ES cell colonies were not washed off. Two hours prior to dissociation, old media was removed and fresh SCML was added. After feeding, the media was removed and the cells were washed with phosphate buffered saline (PBS) that does not contain calcium and magnesium (CMF). The PBS wash was removed, and 0.05% trypsin EDTA was added, followed by incubation for 1-2 minutes. The dish was tapped at 30-second intervals to dislodge the cells. When the cells were free floating in clumps, the trypsin was neutralized with equal volumes of SCML and the mixture was pipetted up and down to generate a single cell suspension.

When the cells were completely dissociated, an aliquot was removed to count, and the remaining cell suspension was pelleted by centrifugation at 1000 rpm for 5 minutes. The supernatant was removed and the cell pellet was resuspended in SCML. The centrifugation and resuspension were repeated to ensure that all the trypsin was removed. After the second SCML wash, the cells were resuspended in 20 ml HepEB media and plated in 2×100 mm bacteriology dishes at 1.5×10⁵ cells/ml with a total volume of 10 ml/dish. HepEB media consists of 80% Iscove's Modified Dulbecco's Medium (IMDM) (GIBCO #31980-030); 5% PFHM-II (Protein-Free Hybridoma Medium) (GIBCO #12040-077); 15% FBS (ES-Qualified Fetal Bovine Serum) (individual lots tested); 2 mM L-Glutamine (GIBCO #25030-081); 4×10⁻⁴ Monothioglycerol (Sigma #M-6145); 50 μg/ml L-Ascorbic Acid (A-4034); 300 μg/ml Transferrin (GIBCO #13008-016); and 25 ng/ml Gentamycin (GIBCO #15710-064). The cells were placed in a designated incubator. This was considered day zero (d0) of the experiment.

On days 2 and 4 the embryoid bodies (EBs) were fed. The EB suspension was transferred from both dishes into a 50 ml tube and set aside. 5 ml HepEB media was added to each dish and the dishes were returned to the incubator. The EBs in the 50 ml tube settled to the bottom of the tube within about 10 minutes. When sufficient EBs pooled at the bottom, the supernatant was removed by aspiration and 10 ml HepEB was added. 5 ml of the EB suspension was transferred to the original 2 dishes and returned to the incubator. On day 5 the EB suspension was transferred from the 2×100 mm bacteriology dishes into 3×100 mm Collagen I coated dishes. An additional 5 ml HepEB media was added to each dish so that the total volume was 1 5 ml per dish.

The cells were cultured as follows during the early stage (day 6-8). On day 6 the HepEB media was removed and 15 ml HepI media was added. HepI media consists of 80% Iscove's Modified Dulbecco's Medium (IMDM) (GIBCO #31980-030); 5% PFHM-II (Protein-Free Hybridoma Medium) (GIBCO #12040-077); 15% FBS (ES-Qualified Fetal Bovine Serum) (individual lots tested); 2 mM L-Glutamine (GIBCO #25030-081); 4×10⁻⁴ Monothioglycerol (Sigma #M-6145); 50 μg/ml L-Ascorbic Acid (A-4034); 10 μM Nicotinamide (Sigma #N-0636); 10 ng/ml EGF murine recombinant (BD #354001); 10 ng/ml Acidic FGF (human recombinant fibroblast growth factor (GIBCO #13241-013); and 25 ng/ml Gentamycin (GIBCO #15710-064). The EBs were attached to the collagen I matrix and had begun to spread out.

On day 7 the old media was removed and 15 ml fresh HepI media was added. The EBs were very spread and some were touching each other. On day 8, the old media was removed and cells were washed with 10 ml PBS/CMF. The PBS was removed and 2 ml 0.05% trypsin EDTA added, followed by an incubation for 1-2 minutes, while tapping the dish at 30-second intervals. When cells and EB clumps were free floating, the trypsin was neutralized with 8 ml HepI and the mixture was pipetted up and down to dissociate the cells into a single cell suspension. 10 ml of cell suspension was transferred to a conical tube (all dishes for each cell line were pooled together). Cells were counted and then centrifuged at 1000 rpm for 5 minutes. The supernatant was discarded and the cells were plated at 2×10⁶ cells per 100 mm Collagen I dish in HepI with a total volume of 15 ml. At least 4×100 mm dishes were plated per cell line. The remaining cells were frozen in HepI freezing media.

The cells were cultured as follows during the middle stage (days 9-11). On days 9 and 10, cells were examined and observations recorded. On day 9, the HepI media was removed and the cells were fed with 15 ml HepII media. HepII media consisted of 80% Iscove's Modified Dulbecco's Medium (IMDM) (GIBCO #31980-030); 5% PFHM-II (Protein-Free Hybridoma Medium) (GIBCO #12040-077); 15% FBS (ES-Qualified Fetal Bovine Serum) (individual lots tested); 2 mM L-Glutamine (GIBCO #25030-081); 4×10⁻⁴ Monothioglycerol (Sigma #M-6145); 0.1 mM MEM Non-Essential Amino Acids (GIBCO #11140-050); 10 μM Nicotinamide (Sigma #N-0636); 10 ng/ml EGF murine recombinant epidermal growth factor (BD #354001); 10 ng/ml Acidic FGF human recombinant fibroblast growth factor (GIBCO #13241-013); 25 ng/ml HGF/SF human recombinant hepatocyte growth factor (BD #354103); and 25 ng/ml Gentamycin (GIBCO #15710-064). Cells formed a monolayer, around 60-70% confluent.

On day 11, the HepII was removed and the cells were washed with 10 ml PBS. PBS was removed and 2 ml 0.04% trypsin EDTA added, followed by an incubation for 1-2 minutes. When cells became free-floating (assisted by tapping the dish), 8 ml HepII was added to neutralize the trypsin, and the solution was pipetted up and down to dissociate the cells into a single cell suspension for transfer into a conical tube. Cells were counted and then centrifuged at 1000 rpm for 5 minutes. The supernatant was discarded and cells were plated 2×10⁶ cells per 100 mm Collagen I dish and at least 4 dishes were plated. The remaining cells were frozen in HepII freezing media.

The cells were cultured as follows during the late stage (days 12-14). On days 12 and 13, the cells were examined and observations recorded. On day 12, the old media was removed and cells were fed with 15 ml HepIII media per 100 mm dish. HepIII media consisted of 80% Iscove's Modified Dulbecco's Medium (IMDM) (GIBCO #31980-030); 5% PFHM-II (Protein-Free Hybridoma Medium) (GIBCO #12040-077); 15% FBS (ES-Qualified Fetal Bovine Serum) (individual lots tested); 2 mM L-Glutamine (GIBCO #25030-081); 4×10⁻⁴ Monothioglycerol (Sigma #M-6145); 0.1 mM MEM Non-Essential Amino Acids (GIBCO #11140-050); 10 μM Nicotinamide (Sigma #N-0636); 10 ng/ml EGF murine recombinant epidermal growth factor (BD #354001); 25 ng/ml HGF/SF human recombinant hepatocyte growth factor (BD #354103); 10 ng/ml OSM murine oncostatin M (R&D Systems #495-MO-025); 100 nM Dexamethasone (Sigma #D-8893); 1×ITS insulin-transferrin-selenium-G (insulin 10 μg/ml, transferrin 5 μg/ml, selenium 5 ng/ml;GIBCO #41400-045); and 25 ng/ml Gentamycin (GIBCO #15710-064). Cells were generally flatter, more epithelial-like in morphology and 60-70% confluent.

On day 14, old media was removed, and the cells were washed with 10 ml PBS. 2 ml 0.05% trypsin was added followed by an incubation for 1-2 minutes. When cells became free-floating (after tapping the dish) 8 ml HepIII was added and pipetted up and down to dissociate into a single cell suspension for transfer into a conical tube. Cells were counted and centrifuged at 1000 rpm for 5 minutes. Supernatant was discarded and plated at 3×10⁶ cells per 100 mm collagen I dish, and 2×10⁵ cells per well in a 24-well collagen I dish in HepIII. As many 24-well dishes were plated as needed for assays.

The cells were cultured as follows during the maturation and selection stage (days 15-18), i.e., stage during which the hepatocyte-like cells are enriched. On days 15 and 16, the cells were examined and observations were recorded. Old media was removed and 15 ml HepIV medium added. Hep IV medium consisted of 90% DMEM without glucose (GIBCO #11966-025); 10% FBS (ES-Qualified Fetal Bovine Serum) (GIBCO #10439-024); 2 mM L-Glutamine (GIBCO #25030-081); 4×10⁻⁴ Monothioglycerol (Sigma #M-6145); 0.1 mM MEM Non-Essential Amino Acids (GIBCO #11140-050); 10 μM Nicotinamide (Sigma #N-0636); 1 mM Pyruvic Acid (P-4562); 10 ng/ml EGF murine recombinant epidermal growth factor (BD #354001); 25 ng/ml HGF/SF human recombinant hepatocyte growth factor (BD #354103); 10 ng/ml OSM murine oncostatin M (R&D Systems #495-MO-025); 100 nM Dexamethasone (Sigma #D-8893); 25 ng/ml Gentamycin (GIBCO #15710-064); and 5 mM Sodium Butyric Acid (B-5887). At this stage, cells began to look like they were dying. The glucose-free media selects for cells that are capable of undergoing gluconeogenesis using pyruvate as the substrate. The sodium butyrate will generally not be detrimental to hepatocytes but will generally be lethal to other cell types.

On day 17 and 18 there was much cell debris from cell death due to selection in glucose-free and sodium butyrate media. On day 17, the old media was removed and cells were washed with 10 ml PBS. Following the PBS wash, cells were fed with 15 ml HepIV. On day 19 and 20, cells were examined and observations were recorded. Cells were washed with 10 ml PBS then fed 15 ml HepIII. Cell morphology was generally very flat and cuboidal in shape. At this stage, the cells are ready to use in assays or, to improve metabolism, induction agents can be used prior to assays.

For induction of hepatic metabolism, old media was removed and Hep III that contains 100 nM Pregnenolone 16α-carbonitrile was added. The next day old media was removed and fresh Hep V was added. Hep V medium consisted of 90% Williams Media E (GIBCO #12551-032); 10% FBS (ES-Qualified Fetal Bovine Serum) (GIBCO #10439-024); 2 mM L-Glutamine (GIBCO #25030-081); 4×10⁻⁴ Monothioglycerol (Sigma #M-6145); 10 μM Nicotinamide (Sigma #N-0636); 10 ng/ml OSM murine oncostatin M (R&D Systems #495-MO-025); 100 nM Dexamethasone (Sigma #D-8893); 1× ITS insulin-transferrin-selenium-G (GIBCO #41400-045); and 25 ng/ml Gentamycin (GIBCO #15710-064).

### Example 3

### Hepatopoietin Stimulates Proliferation of Cells Differentiating from ES Cells into Hepatocyte-like Cells

This Example describes that the addition of mouse hepatopoietin during the middle, late and/or maturation and selection stages stimulates the growth of the cells that are differentiating into hepatocyte-like cells.

Mouse ES cells were differentiated into hepatocyte-like cells as described above, with the addition of 50 ng/ml mouse hepatopoietin protein to the HepII, HepIII and/or HepIV media. The mouse hepatopoietin protein consisted of a portion of the wild-type protein, which portion has the amino acid sequence set forth in SEQ ID NO: 16.

A nucleic acid encoding the protein was obtained as follows. Two primers were synthesized based on the nucleotide sequence of mouse augrnenter of liver regemeration (Alr) mRNA set forth in GenBank® Accession number AF148688 (SEQ ID NO: 15). The 5′ primer used had the sequence 5′ tattcatATGCGGACCCAGCAGAAGCGGGACAT 3′ (SEQ ID NO: 23) and consisted of an HPO sequences (indicated in large caps) linked 5′ to an NdeI site (indicated in italics). The 3′ primer used had the sequence 5′ ttatcaCTAGTCACAGGAGCCGTCCTTCCAT 3′ (SEQ ID NO: 24) and consited of an HPO sequence (indicated in large caps) linked 5′ to two stop codons. These primers were used to amplify mouse HPO from mouse liver cDNA. Since the clone obtained from this amplification contained a mutation at the 3′ end relative to the sequence in GenBank® accession number AF148688, this clone was amplified again using the same 5′ end primer described above and the following 3′ end primer: 5′ TCACTAGTCACAGGAGCCGTCCTTCCATCCGT 3′ (SEQ ID NO: 25). This PCR fragment was subsequently cloned into into pCR 2.1 TOPO vector according to the manufacturer's protocol. Several hundred white colonies were retrieved from Amp/LB plates. Three clones (#1-3) were sequences and a sequence alignment with AF148688 indicated that each clone contained the expected sequence.

The final clone contained the following insert:

| | |
|---|---|
| 5′CATATGCGGACCCAGCAGAAGCGGGACATCAAGTTTAGGGAGGACTG | (SEQ ID NO: 26) |
| TCCGCAGGATCGGGAAGAATTGGGTCGCCACACCTGGGCTTTCCTCCA | |
| TACGCTGGCCGCCTATTACCCGGACAGGCCCACGCCAGAACAACAACA | |
| GGATATGGCCCAGTTCATACATATATTTTCCAAGTTTTACCCCTGCGAG | |
| GAATGTGCGGAAGACATAAGGAAGAGGATAGGCAGGAACCAGCCAGAC | |
| ACAAGCACTCGAGTATCCTTCAGCCAGTGGCTGTGCCGCCTGCACAAT | |
| GAGGTGAATCGGAAGCTGGGCAAGCCTGATTTTGACTGCTCGAGAGTA | |
| GATGAGCGTTGGCGTGACGGATGGAAGGACGGCTCCTGTGACTAGTGA | |
| AAGGGCGAATTCTGCAGATATCCATCACACTGGCGGCCGC | |

The NdeI site and the NotI site are underlined. The HPO coding sequence is indicated in bold and codes for the following 125 amino acid protein:

| | |
|---|---|
| MRTQQKRDIKFREDCPQDREELGRHTWAFLHTLAAYYPDRPTPEQQQDMAQFI | (SEQ ID NO: 16) |
| HIFSKFYPCEECAEDIRKRIGRNQPDTSTRVSFSQWLCRLHNEVNRKLGKPDFDC | |
| SRVDERWRDGWKDGSCD. | |

The purified hepatopoietin protein used in the in vitro differentiation cultures was prepared as follows. The insert of the above-described clone was subcloned into pET23b(+) (Novagen) between the NdeI and NotI sites. This construct was named pMCG204. pMCG204 was transformed into BL21(gold)DE3 cells (Stratagene) and Origami(DE3) cells (Novagen) pursuant to the manufacturer's instructions. Single colonies were inoculated in 25 ml 2×YT media with 100 μg/ml carbenicillin for BL21(gold)DE3 strain clones or 2×YT media with 100 μg/ml carbenicillin, 15 μg/ml kanamycin, and 12.5 μg/ml tetracycline for Origami(DE3) strain clones. Cultures were grown overnight at 37° C. with shaking. 23 ml of the overnight culture was used to inoculate 1 liter of LB broth with appropriate antibiotic(s) in 2.8 liter tri-baffled Fernbach flasks. These were grown at 37° C. with shaking until the O.D. 600 reached 0.75 and then induced with IPTG to a final concentration of 0.6 mM. Growth was continued overnight at 37° C. and cell pellets were harvested the next day.

The cell pellets from either strain were resuspended in 50 mM NaAcetate, pH 5.2, 5 mM DTT, and 1 tablet Complete-EDTA protease inhibitor (Roche) per 25 ml buffer. Lysis was achieved by sonication with 12 ml buffer per 1 liter of cell paste. The cell lysate was then spun at 15,000 rpm in a Sorvall RC 5B plus centrifuge in an SS34 rotor for 20 minutes at 4° C. Supernatant was applied to a 1 ml HiTrap SP XL column (Pharmacia) equilibrated with buffer A (50 mM NaAcetate, pH5.2, 5 mM DTT). The column was washed with several column volumes of buffer A and protein was eluted with a gradient from 0 to 100% buffer B (buffer A with 1 M NaCl) over 25 ml.

Fractions containing soluble HPO were pooled and concentrated with a Centriplus 3,000 NMWCO membrane device. The concentrate was applied to a size exclusion chromatography on Superdex 75 prep grade HiLoad 16/60 column (Pharmacia) previously equilibrated with PBS (Gibco Catalog #: 14190-136). Fractions containing HPO were pooled.

The results of the in vitro differentiation of ES cells with the presence of mouse HPO in the HepII, HepII and/or HepIV media indicate that the presence of HPO stimulates the proliferation of cells giving rise to hepatocyte-like cells, thereby resulting in populations of cells having a higher percentage of hepatocyte-like cells. The presence of hepatopoietin in either medium had this effect, but the strongest effect was seen when it was included in all three media, i.e., during the middle stage, the late stage and the maturation and selection stage. In this case, it appeared that over 90% of the cells in the culture were hepatocyte-like cells.

### Example 4

### Phenotypic Characteristics of Hepatocyte-like Cells and Precursors Thereof

The expression of several genes was monitored during the differentiation of ES cells into hepatocyte-like cells. The genes included α-fetoprotein; γ-glutyryltransferase; hepatocyte nuclear factor (HNF) 1α; HNF 1β; HNF 3α; HNF 3β; HNF 4; albumin; anti-trypsin; transthyretin and cystic fibrosis transmembrane conductance regulator (CFTR).

The level of expression of these genes was monitored by quantitative reverse transcription polymerase chain reaction (RT-PCR) using the primers set forth in Table II:

TABLE II

PCR primers for hepatocyte-specific
markers (GIBCO-BRL)

| | | |
|---|---|---|
| Marker | Forward Primer | Reverse Primer |

| | | |
|---|---|---|
| α-Fetoprotein | CAGCCAAAGTGGAGTGGAAAGA | AACTCTCGGCAGGTTCTGGAA |
| | (SEQ ID NO:27) | (SEQ ID NO:28) |
| γ-Glutyryl- | ATTGAGAAGACCCCTGCCTTGT | ATCTGCAATGTGTCAGCCAGC |
| transferase | (SEQ ID NO:29) | (SEQ ID NO:30) |
| HNF1α | ATTGAGAAGACCCCTGCCTTGT | ATCTGCAATGTGTCAGCCAGC |
| | (SEQ ID NO:31) | (SEQ ID NO:32) |
| HNF1β | CCTGAACCAATCCCACCTCTCT | ATCTCCCGTIGCTTTCTGACG |
| | (SEQ ID NO:33) | (SEQ ID NO:34) |
| HNF3α | ATTGAGAAGACCCCTGCCTTGT | ATCTGCAATGTGTCAGCCAGC |
| | (SEQ ID NO:35) | (SEQ ID NO:36) |
| HNF3β | AAGAAGATGGCTTTCAGGCCC | AAGGCCATTGAAGTGTGGTGG |
| | (SEQ ID NO:37) | (SEQ ID NO:38) |
| HNF4 | GACTCTCTAAAACCCTTGCCGG | CCATGGTCAACACCTGCACAT |
| | (SEQ ID NO:39) | (SEQ ID NO:40) |
| Albumin | CGCCCATCGGTATAATGATTTG | CTGCACTAATTTGGCATGCTCA |
| | (SEQ ID NO:41) | (SEQ ID NO:42) |
| Anti-Trypsin | TGCTTGATGTGCACCATTGC | TGCTCCAGATGCTGCATCTTC |
| | (SEQ ID NO:43) | (SEQ ID NO:44) |
| Transthyretin | AAGCAGAGTGGACCAACCGTT | AAGCAGAGTGGACCAACCGTT |
| | (SEQ ID NO:45) | (SEQ ID NO:46) |
| CFTR | TTAATGTGCTTGGCCCGATC | CCAGCGAAGGCTTGTTTTAGAA |
| | (SEQ ID NO:47) | (SEQ ID NO:48) |

The results are set forth in Table III (wherein “X” represents that expression was detected), and show that some genes are expressed from day 3 to day 29 of the culture, whereas others are expressed specifically at certain stages of differentiation, as expected.

TABLE III
RT-PCR results for ES cell-derived hepatocytes

| | Days of In Vitro Differentiation | | | | | | | | | | | | | | | | |
|---|---|---|---|---|---|---|---|---|---|---|---|---|---|---|---|---|---|
| Markers | 3 | 4 | 5 | 6 | 7 | 8 | 9 | 10 | 11 | 12 | 15 | 17 | 19 | 22 | 24 | 26 | 29 |
| α-fetoprotein | X | X | X | X | X | X | X | X | X | X | X | X | X | | X | X | X |
| γ-glutyryltransferase | X | X | X | X | X | X | X | X | X | X | | | | | X | | X |
| HNF 1α | X | X | X | X | X | X | X | X | X | X | | | | | X | | X |
| HNF 1β | X | X | X | X | X | X | X | X | X | X | X | | X | | X | | X |
| HNF 3α | X | X | X | X | X | X | X | X | X | X | | | | | X | | X |
| HNF 3β | X | X | X | X | X | X | X | X | X | X | X | | X | | X | | X |
| HNF 4 | X | X | X | X | X | X | X | X | X | X | | | | | | | X |
| Albumin | | | | | | X | X | X | X | X | | | | | | | |
| Anti-trypsin | | | | | | X | X | X | X | X | | | | | | | X |
| Transthyretin | X | | X | X | X | X | X | X | X | X | | | | | | | X |
| CFTR | X | | | | | X | X | X | X | X | | | | | | | X |

To further characterize the hepatocyte-like cells, cytochrome p450 proteins were detected by immunohistochemical detection. Differentiated hepatocyte-like cells were fixed with 4% paraformaldehyde, treated with specific goat anti-rat CYP sera recognizing CYP1A1, CYP2B1, CYP2C6, CYP2C11, CYP2C13, CYP3A2 or CYP4A1 (Daiichi Pure Chemical Co. LTD, Tokyo, Japan), washed, and the treated with an alkaline phosphatase-labeled rabbit anti-goat antibody, according to methods known in the art. Labeled cells were detected with the alkaline phosphatase substrate NBT/BCIP. After treatment, cells were conterstained with eosin. The results indicate the presence of all of the CYPs tested, with higher levels of CYP1A1, CYP2B1 and CYP2C6, as is seen in primary cultures of differentiated hepatocytes. Thus, these results confirm that the differentiated cells obtained from ES cells have characteristics of differentiated hepatocytes.

### Example 5

### Functional Characteristics of Hepatocyte-like Cells

The presence of CYP proteins in the hepatocyte-like cells was also determined by monitoring the conversion of certain compounds added to the hepatocyte-like cells. Two test compounds (7-ethoxy-coumarin and dextromethorphan) were added to hepatocyte-like cells. The supernatants of the cells were then subjected to high pressure liquid chromatography (HPLC) and mass spectrometry to identify products of the conversion of the test compounds by CYP3a, CYP2d, CYP2e1 and CYP1a2. The expected conversion products were obtained in each case, thereby indicating that these cytochrome p450 enzymes are present and finctionally active in the hepatocyte-like cells.

Another test that was used to characterize the hepatocyte-like cells is the dibenzylfluorescein (DBF) assay for metabolic activity. See, for example, Stresser et al., Drug Metab. Disp., 28:1440-48 (2000). These assays were conducted using a DBF compound (Molecular Probes, Eugene Oreg.) according to the manufacturer's recommendations. The results indicate that the test was positive, and that similar results were obtained with hepatocyte-like cells obtained from differentiation in the presence or in the absence of HPO.

Equivalents

While specific embodiments of the subject invention have been discussed, the above specification is illustrative and not restrictive. Many variations of the invention will become apparent to those skilled in the art upon review of this specification. The appended claims should be interpreted by reference to the claims, along with their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

1. A method for obtaining a hepatocyte-like cell, comprising:
a) providing a stem cell;
b) culturing the stem cell in a first medium comprising effective amounts of an acidic fibroblast growth factor (aFGF) and an epidermal growth factor (EGF) for about 2 to 4 days to obtain a first cell population;
c) culturing a cell of the first cell population in a second medium comprising an effective amount of hepatocyte growth factor (HGF) for about 2 to 4 days to obtain a second cell population; and
d) culturing a cell of the second cell population in a third medium comprising effective amounts of oncostatin-M for about 2 to 4 days to obtain a third cell population, the third cell population comprising a plurality of hepatocyte-like cells.

2. The method of claim 1, wherein the aFGF is present at a concentration of about 1-20 ng/ml and the EGF is present at a concentration of about 1-20 ng/ml in the first medium.

3. The method of claim 1, wherein the HGF is present at a concentration of about 5-50 ng/ml in the second medium.

4. The method of claim 1, wherein the oncostatin-M is present at a concentration of about 1-30 ng/ml in the third medium.

5. The method of claim 1, further comprising:
e) culturing a plurality of cells of the third cell population in a medium suitable for selectively culturing gluconeogenic cells, thereby obtaining a cellular composition comprising an enriched population of hepatocyte-like cells.

6. The method of claim 5, further comprising:
f) culturing a plurality of cells of the enriched population of hepatocyte-like cells in a fifth medium suitable for stimulating hepatocyte-associated metabolic functions, thereby obtaining a cellular composition comprising a population of hepatocyte-like cells having enhanced hepatocyte-associated metabolic activity.

7. The method of claim 6, further comprising:
g) culturing a hepatocyte-like cell in a sixth medium comprising nicotinamide, oncostatin M, dexamethasone, insulin, transferrin and selenium.

8. The method of claim 1, further comprising:
e) culturing a plurality of cells of the third cell population in a medium suitable for inducing hepatocyte-like metabolic functions, thereby obtaining a cellular composition comprising a population of hepatocyte-like cells having enhanced hepatocyte-like metabolic activity.

9. A method for obtaining a hepatocyte-like cell, comprising:
a) providing an ES cell;
b) stimulating the differentiation of the ES cell into embryoid bodies for about 5 days;
c) culturing the embryoid bodies in a first medium comprising effective amounts of an aFGF and an EGF for about 1 to 2 days to obtain embryoid bodies;
d) dissociating the embryoid bodies into a single cell suspension and culturing the single cell suspension for about 1 to 2 days in the first medium to obtain a first cell population;
e) culturing a cell of the first cell population in a second medium comprising an effective amount of EGF, HGF and aFGF for about 2 to 4 days to obtain a second cell population; and
f) culturing a cell of the second cell population in a third medium comprising effective amounts of oncostatin-M, EGF, and HGF for about 2 to 4 days to obtain a third cell population, the third cell population comprising a plurality of hepatocyte-like cells.

10. A cellular composition comprising viable cells, wherein at least 90% of the viable cells are hepatocyte-like cells.

11. The cellular composition of claim 10, wherein the hepatocyte-like cells:
a) use pyruvate as a carbon source;
b) express two or more cytochrome P450 enzymes; and
c) are viable in 5 mM butyric acid.

12. A cellular composition obtained by the method of claim 1.

13. A cellular composition obtained by the method of claim 9.

14. A method for treating a subject in need of liver cells, comprising administering to the subject a therapeutically effective amount of the hepatocyte-like cells of claim 12.

15. A method for treating a subject in need of liver cells, comprising administering to the subject a therapeutically effective amount of the hepatocyte-like cells of claim 13.

16. An isolated nucleic acid encoding a polypeptide comprising the amino acid sequence of SEQ ID NO: 18.

17. An isolated polypeptide comprising the amino acid sequence of SEQ ID NO: 18.
